(19) United States
(12) Patent Application Publication
(10) Pub. No.: US 2002/0155157 A1
(43) Pub. Date: Oct. 24, 2002

(54) Compositions and methods for polynucleotide delivery

(75) Inventors: Dan Luo, Ithaca, NY (US); Mark Muller, Columbus, OH (US)

Correspondence Address: PILLSBURY WINTHROP, LLP, P.O. BOX 10500, MCLEAN, VA, 22102, US

(73) Assignee: The Ohio State University Research Foundation, Columbus, OH

(21) Appl. No.: 09/957,667

(22) Filed: Sep. 21, 2001

Related U.S. Application Data
(63) Continuation of application No. 09/244,722, filed on Feb. 10, 1999, now abandoned.
(60) Provisional application No. 60/074,213, filed on Feb. 10, 1998.

(51) Int. Cl. A61K 48/00; A61K 9/14

(52) U.S. Cl. 424/484; 514/44

(57) ABSTRACT

Compositions are disclosed comprising complexes of polynucleotide molecules which are covalently coupled to ligand moieties that are specifically bound to ligand-binding molecules, where the ligand binding molecules have multiple ligand-binding sites that specifically bind to the ligand moieties. Each polynucleotide molecule in these complexes is covalently coupled to at least one ligand moiety which is specifically bound to a ligand-binding site on a ligand-binding molecule. Most or all of the ligand-binding molecules in the complexes are linked to multiple polynucleotide molecules by specific binding to multiple ligand moieties; and most or all of the polynucleotide molecules in the compositions are included in these complexes. Also disclosed are methods for preparing compositions of the invention and for using them to introduce polynucleotides into cells, including for expressing genes for gene therapy. Compositions comprising biotinylated double-stranded DNA molecules in complexes with neutral avidin are exemplified.

### BACKGROUND OF THE INVENTION

1. Field of the Invention

The present invention relates to compositions and methods for delivery of polynucleotides into viable cells, particularly mammalian cells. More particularly, the invention relates to complexes which comprise polynucleotides covalently coupled to ligand moieties, which ligand moieties are specifically bound to ligand-binding sites of ligand-binding molecules. The invention relates still more particularly to such complexes in which each polynucleotide molecule is covalently coupled to multiple ligand moieties, each ligand-binding molecule comprises multiple ligand-binding sites for those ligand moieties, and the number of polynucleotide molecules coupled to ligand moieties which are specifically bound to ligand-binding molecules in the complexes is equal to a majority of the ligand-binding sites of the ligand-binding molecules in the complexes.

2. Description of Related Art

Liposome-mediated Intracellular Delivery of Polynucleotides

The process of introduction (or “delivery”) of polynucleotides into cells is variously called “transformation” (the most general term), “transfection” (primarily for infectious viral genomes, but also sometimes used interchangeably with “transformation”), or “transduction” (for transfer of a non-viral gene via a viral vector). One of the first studies on transfer of functional DNA into mammalian cells, which showed the transfer of a purified herpes virus thymidine kinase gene to cultured mouse cells, was reported over three decades ago. Wigler, M., Silverstein, S., Lee, L. S., Pellicer, A., Cheng, Yc., Axel, R. Cell 11:223-232 (1977). Since then, variety of compositions and methods have been developed for delivering polynucleotides into mammalian cells for therapeutic purposes, including anti-sense oligonucleotides as well as constructs for gene expression.

For instance, appearance of beta-lactamase activity in cultured animal cells upon liposome-mediated transfer of a bacterial gene was demonstrated within a few years of the initial reports on transformation of mammalian cells. Wong, T. K., Nicolau, C. and Hofschneider P. H., Gene 10:87-94 (1980). In vivo transfection by vector derived from a viral genome, using a liposome vehicle, also has been disclosed. Brigham, K. L., Meyrick, B., Christman, B., Magnuson, M., King, G., Berry, L. C., Jr., Am J Med. Sci. 298:278-281(1989). These authors reported successful in vivo transfection of lungs of mice with a gene encoding the intracellular enzyme, chloramphenicol acetyltransferase (CAT). Transfection was accomplished by injecting a plasmid containing the coding region for CAT driven by the SV40 early promoter (pSV2CAT) complexed to specially synthesized cationic liposomes. Intravenous or intratracheal injection of DNA-liposomes resulted in expression of the CAT gene in the lungs, persisting for at least a week, with little enzyme activity detectable in systemic organs.

More recently, Song, Y. K., Liu, F., Chu, S. and Liu, D., Hum Gene Ther 8:1585-1594 (1997), discloses characterization of cationic liposome-mediated gene transfer in vivo by intravenous administration. Using a cytomegalovirus (CMV)-driven gene expression system containing either the luciferase or green fluorescence protein gene as a reporter and two cationic lipids [N-(2,3-dioleoyloxy)propyl-N,N,N-trimethylammonium chloride (DOTMA) and 1,2-dioleoyloxy-3-trimethylammonium propane (DOTAP)], it was demonstrated in vivo by a single intravenous injection of DNA/liposome complexes into mice, that cationic liposomes are capable of transfecting cells in organs such as the lung, heart, liver, spleen, and kidney.

Delivery of a PCR amplified DNA fragment into cells recently has been demonstrated, using anionic liposomes, as a model for the use of synthetic genes for gene therapy. Li, S., Brisson, M., He, Y. and Huang, L., Gene Ther 4:449-454 (1997). PCR amplified fragments were used as a model to test the feasibility of using synthetic genes for gene therapy. The CAT reporter gene driven by the CMV promoter (CMV-CAT), i.e., a nuclear expression system, or by the bacteriophage T7 promoter (T7-CAT), i.e., a cytoplasmic expression system, was used to evaluate this concept. The expression efficiency of both plasmids (pUCCMV-CAT and pT7-CAT) and their corresponding linear PCR fragments (fCMV-CAT and fT7-CAT) were compared on a molar basis. Limited expression of CAT was found with the linear fCMV-CAT construct which requires nuclear expression. However, under conditions of cytoplasmic expression, linear fT7-CAT consistently gave a CAT activity comparable to that of circular pT7-CAT.

Delivery of Polynucleotides Using Electroporation

Another technique used for introducing polynucleotides into cells is known as “electroporation.” See, for instance, “Optimizing electroporation parameters for a variety of human hematopoietic cell lines,” McNally, M. A., Lebkowski, J. S., Okarma, T. B. and Lerch, L. B., Biotechniques 6:882-886 (1988). The parameters affecting electroporation of four human hematopoietic cell lines were investigated. The optimal conditions for electroporation were described for both transient and stable expression of foreign genes. A correlation was show to exist between the levels of transient gene expression and stable transfection frequency. In addition, in this system linear DNA yielded higher stable transfection frequencies than supercoiled DNA.

Delivery Using Polynucleotide-coated Micro-particles

In recent years, “naked” DNA vaccines have been developed using various micro-particles as carriers. For instance, Tang, D. C., DeVit, M. and Johnston, S. A., have disclosed “genetic immunization” as a simple method for eliciting an immune response. Nature 356:152-154 (1992). The authors reported that an immune response can be elicited by introducing the gene encoding a protein directly into the skin of mice. This was achieved using a hand-held form of the “biolistic” system which can propel DNA-coated gold microprojectiles directly into cells in the living animal.

Similarly, protection of ferrets against influenza challenge with a DNA vaccine to the haemagglutinin has been reported. Webster, R. G., Fynan, E. F., Santoro, J. C., and Robinson, H., Vaccine 12:1495-1498 (1994). Delivery of DNA-coated gold beads by “gene gun” to the epidermis was reported to be much more efficient than intramuscular delivery of DNA in aqueous solution. The antibody response induced by DNA delivered by gene gun was said to be more cross-reactive than DNA delivered in aqueous solution or after natural infection.

High-efficiency gene transfer, including in vivo gene transfer, using a technique called “electroporation” also have been reported. See, for example, Nishi, T. et al., Cancer Res. 56:1050-1055 (1996).

Enhanced Delivery of Naked Polynucleotides by Non-covalent Complexes

Direct gene transfer into mammalian cells in vivo has been disclosed, for instance, in mouse muscle. Wolff, J. A., Malone, R. W., Williams, P., Chong, W., Acsadi, G., Jani, A. and Feigner, P. L., Science 247:1465-1468 (1990). In this study, RNA and DNA expression vectors containing genes for chloramphenicol acetyltransferase, luciferase, and beta-galactosidase were separately injected into mouse skeletal muscle in vivo. Protein expression was readily detected in all cases, and it was reported that no special delivery system was required for these effects. The extents of expression from both RNA and DNA constructs were reported to be comparable to that obtained from fibroblasts transfected in vitro under optimal conditions.

However, in many other studies, uptake of purified polynucleotides, such as “naked” DNA, has been shown to be enhanced by formation of non-covalent, non-specific complexes with various agents, particularly polycations such as diethylaminoethyl-dextran (DEAE-dextran) or poly-L-lysine (see, for instance, Ehrlich, M., Sarafyan, L. P., Myers, D. J., Biochim. Biophys. Acta 54:397409 (1976)) or polyornithine, spermine, or polyarginine (see, for example, Farber, F. E., Melnick, J. L., and Butel, J. S., Biochim Biophys Acta 390:298-311 (1975)). One of the earliest methods for delivery of polynucleotides into mammalian cells was an assay of transforming activity of “naked” tumor virus DNA in mammalian cells using calcium phosphate precipitates of the DNA. van der Eb, A. J. and Graham, F. L., Methods Enzymol. 65:826-839 (1980).

Polyamidoamine cascade polymers have been shown to mediate efficient transfection of cells in culture. Haensler, J. and Szoka, F. C., Jr., Bioconjug. Chem. 4:372-379 (1993). Cascade polymers, also known as Starburst dendrimers, are spheroidal polycations that can be synthesized with a well-defined diameter and a precise number of terminal amines per dendrimer. These workers have shown, using luciferase and beta-galactosidase containing plasmids, that dendrimers mediate high efficiency transfection of a variety of suspension and adherent cultured mammalian cells. Dendrimer-mediated transfection is a function both of the dendrimer/DNA ratio and the diameter of the dendrimer. Maximal transfection of luciferase was obtained using a diameter of 68 Å and a dendrimer to DNA charge ratio of 6/1 (terminal amine to phosphate). Expression was unaffected by lysomotrophic agents such as chloroquine and only modestly affected (2-fold decrease) by the presence of 10% serum in the medium. Cell viability, as assessed by dye reduction assays, decreases by only 30% at 150 micrograms dendrimer/mL in the absence of DNA and about 75% in the presence of DNA. Under similar conditions polylysine caused a complete loss of viability. Gene expression decreased by 3 orders of magnitude when the charge ratio was reduced to 1:1. When GALA, a water soluble, membrane-destabilizing peptide, was covalently attached to the dendrimer via a disulfide linkage, transfection efficiency of the 1:1 complex is increased by 2-3 orders of magnitude. The authors hypothesized that the high transfection efficiency of the dendrimers may not only be due to their diameter and shape but may also be caused by the pka's (3.9 and 6.9) of the amines in the polymer. The low pKa's permit the dendrimer to buffer the pH change in the endosomal compartment.

Wyman, T. B., Nicol, F., Zelphati, O., Scaria, P. V., Plank, C., and Szoka, F. C., Jr., Biochemistry 36:3008-3017 (1997), describes the design, synthesis, and characterization of a cationic peptide that binds to nucleic acids and permeabilizes bilayers. This cationic amphipathic peptide, KALA (WEAKLAKALAKALAKHLA KALAKALKACEA), binds to DNA, destabilizes membranes, and mediates DNA transfection. KALA undergoes a pH-dependent random coil to amphipathic alpha-helical conformational change as the pH is increased from 5.0 to 7.5. One face displays hydrophobic leucine residues, and the opposite face displays hydrophilic lysine residues. KALA-mediated release of entrapped aqueous contents from neutral and negatively charged liposomes increases with increasing helical content. KALA binds to oligonucleotides or plasmid DNA and retards their migration in gel electrophoresis. In cultured cells, KALA assists oligonucleotide nuclear delivery when complexes are prepared at a 10/1 (+/−) charge ratio. KALA/DNA (10/1)(+/−) complexes mediate transfection of a variety of cell lines.

Another new peptide vector for efficient delivery of oligonucleotides into mammalian cells has been described, based on the use of a short peptide vector, termed MPG (27 residues), which contains a hydrophobic domain derived from the fusion sequence of HIV gp41 and a hydrophilic domain derived from the nuclear localization sequence of SV40 T-antigen. Morris, M. C., Vidal, P., Chaloin, L., Heitz, F. and Divita, G., Nucleic Acids Res. 25:2730-2736 (1997). MPG exhibits relatively high affinity for both single- and double-stranded DNA in a nanomolar range. It appears that the main binding between MPG and oligonucleotides occurs through electrostatic interactions, which involve the basic-residues of the peptide vector. Further peptide/peptide interactions also occur, leading to a higher MPG/oligonucleotide ratio (in the region of 20/1), which suggests that oligonucleotides are most likely coated with several molecules of MPG. Premixed complexes of peptide vector with single or double stranded oligonucleotides are delivered into cultured mammalian cells in less than 1 h with relatively high efficiency (90%). The interaction with MPG strongly increases both the stability of the oligonucleotide to nuclease and crossing of the plasma membrane. The mechanism of cell delivery of oligonucleotides by MPG does not follow the endosomal pathway, which explains the rapid and efficient delivery of oligonucleotides in the nucleus.

Alila, H., et al., Hum Gene Ther 8:1785-1795 (1997), discloses expression of biologically active human insulin-like growth factor-I following intramuscular injection of a formulated plasmid in rats. The hIGF-I plasmid, formulated as a complex with PVP, produced a localized and sustained level of biologically active hIGF-I.

Polynucleotide Delivery Complexes Using Receptor-mediated Endocytosis

Receptor-mediated gene delivery and expression in vivo has been reported using cations such as polylysine covalently coupled to various receptor ligands to provide a means for attaching polynucleotides to those ligands via formation of non-covalent complexes with the attached cation. See, for instance, Wu, G. Y. and Wu, C. H., J. Biol. Chem. 263:14621-14624 (1988). In this work, a soluble DNA carrier system was used to target a foreign gene specifically to liver in vivo, via asialoglycoprotein receptors. The DNA carrier consisted of a galactose-terminal (asialo-)glycoprotein, asialoorosomucoid (AsOR), covalently linked to poly-L-lysine. The conjugate was complexed in a 2:1 molar ratio (based on AsOR content of the conjugate) to the plasmid, pSV2 CAT, containing the gene for the bacterial enzyme chloramphenicol acetyltransferase (CAT). Intravenous injection of [³²P]plasmid DNA complexed to the carrier demonstrated specific hepatic targeting with 85% of the injected counts taken up by the liver in 10 min compared to only 17% of the counts when the same amount of [³²P]DNA alone was injected under identical conditions. Homogenates of livers taken 24 h after injection of the complex revealed that the targeted CAT gene was functional as reflected by the detection of CAT activity. Assays for CAT activity in other organs (spleen, kidney, lungs) failed to demonstrate any activity in these organs.

In addition, biochemical and functional analysis of an adenovirus-based, polylysine-containing ligand complex for receptor-mediated gene transfer has been described. See, for instance, Fisher, K. J. and Wilson, J. M., Biochem. J. 299:49-58 (1994). This study was based on the observations that, although a significant percentage of the plasmid-based DNA complex is lost to lysosomal degradation following receptor-mediated endocytosis, simultaneous infection with adenovirus has been shown to increase the level of transgene expression [Curiel, Agarwal, Wagner and Cotten (1991) Proc. Natl. Acad. Sci. U.S.A. 88, 8850-8854; Wagner, Zatloukal, Cotten, Kirlappos, Mechtler, Curiel and Birnstiel (1992) Proc. Natl. Acad. Sci. U.S.A. 89, 6099-6103]. The paper describes an adenovirus-based ligand complex where the plasmid DNA, polycation-ligand conjugate and adenovirus are contained within a single particle structure. At the core of the transfection particle is a replication-defective recombinant adenovirus encoding a cDNA minigene for human placenta alkaline phosphatase that was chemically modified with poly(L-lysine) (Ad-pLys). Electron microscopy of an adenovirus-based ligand complex formed by successively adding plasmid DNA and an asialo-orosomucoid-poly(L-lysine) conjugate to Ad-pLys revealed structures that appeared as intact viral particles coated with a dense biomolecular layer. Adenovirus-based ligand complexes containing either a luciferase or beta-galactosidase reporter plasmid were shown to efficiently deliver the plasmid transgene to cells that express the hepatic asialoglycoprotein receptor. Furthermore, the poly(L-lysine) modification greatly reduced the infectivity potential of the virus without causing a concomitant loss of augmented gene transfer. As an alternative to infectious virions, incomplete products of viral assembly were also considered as a source for endosomalytic activity. However, these defective virions were unable to significantly enhance plasmid transgene delivery.

Receptor-mediated gene delivery employing lectin-binding specificity also has been reported. See, for instance, Batra, R. K., Wang-Johanning, F., Wagner, E., Garver, R. I., Jr., Curiel, D. T., Gene Ther. 1:255-260 (1994). Given that malignant cells can be distinguished from normal by differences in the expression of cell surface carbohydrates, these authors hypothesized that transductional targeting would be feasible by molecular conjugate vectors which achieve cell binding by virtue of lectins directed against the cell surface glycocalyx. They have shown that gene transfer can be accomplished by these novel lectin-targeted molecular conjugate vectors. This same group has shown that similar molecular conjugate vectors mediate efficient gene transfer into gastrointestinal epithelial cells. Batra, R. K., Berschneider, H. and Curiel, D. T., Cancer Gene Ther. 1:185-192 (1994). The authors were able to achieve efficient transfection of transformed (Caco2 cells) and nontransformed gastrointestinal cells derived from neonatal piglets utilizing molecular conjugate vectors. Analysis of heterologous gene expression revealed that enterocytes could serve as a secretory cellular source of alpha 1-antitrypsin and factor IX. Transient expression of heterologous DNA persisted for up to 2 weeks following transfection.

Polynucleotide Delivery Using Biotin and/or a Biotin-binding Protein

Enhancement of cellular uptake of a biotinylated antisense oligonucleotide or a peptide, mediated by a biotin-binding protein, avidin, has been disclosed. Pardridge, W. M., Boado, R. J., FEBS Lett 288:30-32 (1991). The authors reported that the cellular uptake of a model antisense oligonucleotide of 21 bases, biotinylated at one end, was markedly stimulated by the presence of avidin which is a cationic protein. Conversely, the bacterial homologue of avidin, streptavidin, which is a slightly acidic protein, did not facilitate cellular uptake. The avidin-mediated uptake of biotinylated derivatives was competitively inhibited by another cationic protein, protamine, with a Kᵢ of 5 micrograms/ml; was saturable, temperature- and time-dependent; and was associated with endocytosis. The authors reported that in one experiment the uptake by isolated brain capillaries of [³²P]bio-antisense oligonucleotide was increased about four fold by a vast excess of avidin. Id. at page 32, col. 1, referring to FIG. 1D.

Complete protection of antisense oligonucleotides against serum nuclease degradation by an avidin-biotin system has been reported by these same authors. Boado, R. J., Pardridge, W. M., Bioconjug. Chem 3:519-523 (1992). In this study, 21-mer antisense oligonucleotides complementary to nucleotides 162-182 and 161-181 of the bovine GLUT1 glucose transporter mRNA were synthesized with a 6-aminodeoxyuridine at positions 3 and 20, respectively, biotinylated with NHS- or NHS-XX-biotin to yield near 5′- or near 3′-biotinylated oligonucleotide (bio-DNA), and 5′- and 3′-end radiolabeled. Serum induced a rapid degradation of unprotected (no avidin) [5′-³²P]-5′-bio-DNA (>95% at 30 min). Avidin partially protected this construct (approximately 31% of intact 21-mer oligo remained at 1 h). Similar results were obtained with the [3′-³²P]-5′-bio-DNA; however, no degradation products of varying size were observed, confirming that the degradation is mediated primarily by a 3′-exonuclease. Incubation of the [5′-³²P]-3′-bio-DNA with serum showed a rapid conversion to the 20- and 19-mer forms (t_{1/2} approximately 13 min). Conversely, avidin totally protected this construct against the serum 3′-exonuclease.

In subsequent studies, these same workers reported complete inactivation of target mRNA by biotinylated antisense oligodeoxynucleotide-avidin conjugates. Boado, R. J. and Pardridge, W. M., Bioconjug. Chem. 5:406-410 (1994). They noted that biotinylation of phosphodiester oligodeoxynucleotides (PO-ODN) at the 3′-terminus provides complete protection against serum 3′-exonuclease degradation. This study was undertaken to determine if antisense 3′-biotinylated PO-ODN-avidin constructs are able to recognize and inactivate the target mRNA through RNase H-mediated degradation. A 21-mer antisense PO-ODN complementary to the tat gene encompassing nucleotides 5402-5422 of the HIV-1 genome was synthesized with biotin conjugated to the 3′-terminus (bio-tat). Gel mobility assays using [5′-³²P]-labeled bio-tat ODN and avidin showed that the bio-tat ODN was fully monobiotinylated. Aliquots of [³²P]-labeled sense or antisense tat RNA (337 and 351 nucleotides, respectively) were prepared from transcription plasmids and were preincubated with an excess of bio-tat ODN with or without avidin constructs and digested with RNase H. Products were resolved with sequencing gel and analyzed by autoradiography. Complete conversion to predicted RNA fragments resulting from RNase H digestion of the RNA-ODN duplex (53 and 263 nucleotides) was observed when [³²P]-tat sense RNA was incubated with antisense bio-tat ODN or conjugated to avidin or an avidin-cationized human serum albumin (cHSA) complex. Conversely, no degradation of [³²P]-tat-antisense RNA was observed after incubation with antisense bio-tat ODN and RNase H. In addition, the avidin-cHSA complex significantly increased (84-fold) the uptake of [³²P]-internally labeled bio-tat ODN and its stability against cellular nuclease degradation in peripheral blood lymphocytes.

Use of neutral avidin has been reported to improve pharmacokinetics and brain delivery of biotin per se, when bound to an avidin-monoclonal antibody conjugate. Kang, Y. S. and Pardridge, W. M., J. Pharmacol. Exp. Ther. 269:344-350 (1994). The authors stated that delivery of therapeutic agents through the brain capillary endothelial wall, which makes up the blood-brain barrier (BBB) in vivo, is enabled by coupling drugs to brain drug delivery transport vectors, such as the OX26 monoclonal antibody to the transferrin receptor located on the BBB. They also disclosed that drug conjugation to delivery vectors is possible by the use of avidin/biotin technology, and the production of avidin/vector conjugates potentially allows for the delivery through the BBB of many biotinylated therapeutics. However, the use of avidin was said to cause reduced brain delivery of avidin/vector conjugates, because of the rapid systemic clearance of such conjugates from the bloodstream. The authors further stated that, because previous studies had shown that this rapid elimination is due to avidin's cationic nature, the present studies describe the production of neutral avidin-OX26 antibody conjugates. Isoelectric focusing demonstrated the pls of avidin and neutral avidin were >9 and 5 to 6, respectively. Neutral avidin and the OX26 antibody, which was purified from serum-free hybridoma-conditioned supernatants, were conjugated with a thio-ether linkage. The area under the plasma concentration curve of [³H] biotin/neutral avidin-OX26 was more than 5-fold greater than that for [³H] biotin/avidin-OX26. The mean residence time of [³H] biotin/neutral avidin-OX26 in plasma was 11.3+/−0.2 hr. The BBB permeability-surface area product was not significantly different for either [³H] biotin/neutral avidin-OX26 or [³H] biotin/avidin-OX26. The delivery of [³H] biotin to brain reached 0.20 to 0.25% of injected dose per gram brain by 2-6 hr after single intravenous injection, whereas the brain delivery of [³H] biotin/avidin-OX26 did not exceed 0.05% injected dose per g.

Pharmacokinetics of [³H]biotin bound to different avidin analogues has also been examined in detail. Kang, Y. S., Saito, Y. and Pardridge, W. M., J. Drug Target. 3:159-165 (1995). The authors noted that use of avidin-biotin technology in drug delivery facilitates the conjugation of biotinylated therapeutics to transport vectors that are enabled to undergo receptor-mediated transcytosis through the brain capillary endothelial wall, which makes up the blood-brain barrier (BBB) in vivo. They further noted that the conjugation of avidin, a cationic glycosylated protein, to transport vectors greatly increases the rate of removal of the vector from the bloodstream, owing to rapid uptake of avidin by peripheral tissues such as liver and kidney. However, they suggested that modified avidins may retain high affinity biotin binding properties, but may not be rapidly removed from plasma by peripheral tissues, and such avidin analogues would provide preferred plasma pharmacokinetic profiles. Therefore, these studies investigated the pharmacokinetics of plasma removal of [³H]biotin bound to one of six different avidin analogues: streptavidin, Neutra-lite avidin, avidin, neutral avidin, Lite-avidin, and succinylated avidin. Isoelectric focusing studies showed that avidin and Lite-avidin were highly cationic proteins, whereas neutral avidin, Neutra-lite avidin, and streptavidin were neutral proteins, and succinylated avidin had an acidic isoelectric point. The avidin analogues fell into two groups with respect to rate of biotin removal from plasma. The low clearance group included streptavidin and Neutra-lite avidin, which had a mean plasma clearance of 0.41 mL/min/kg. The high clearance group consisted of succinylated avidin, neutral avidin, and Lite-avidin and had a mean plasma clearance of 17 mL/min/kg, or 40-fold faster than the low clearance avidins.

Pharmacokinetics and organ clearance of a 3′-biotinylated, internally [³²P]-labeled phosphodiester oligodeoxynucleotide coupled to a neutral avidin/monoclonal antibody conjugate has also been investigated. Kang, Y. S., Boado, R. J. and Pardridge, W. M., Drug Metab. Dispos. 23:55-59 (1995). In particular, pharmacokinetics and organ uptake of a 3′-biotinylated, [³P] internally labeled 36-mer phosphodiester oligodeoxynucleotide (PO-ODN) were measured after intravenous injection in the anesthetized adult rat. The PO-ODN was antisense to the tat gene of the human immunodeficiency virus, and was 3′-biotinylated to a) protect against serum and tissue 3′-exonuclease activity, and b) facilitate coupling to a neutral avidin-based transcellular drug delivery vector. The latter was comprised of a covalent conjugate of neutral avidin (NLA) and the OX26 murine monoclonal antibody to the rat transferrin receptor. The PO-ODN was internally labeled at the 21-nucleotide position to prevent rapid hydrolysis [³²P] label by serum and tissue 5′-phosphatases. The uptake of the 3′-bio-[³²P21]PO-ODN by brain, heart, kidney, lung, and liver was measured. The studies showed that the unconjugated 3′-bio-[³²P21]PO-ODN was rapidly removed from plasma. Conjugation of the 3′-bio-PO-ODN to the NLA-OX26 vector reduced the systemic clearance 50%, owing to a >10-fold reduction in renal clearance. Following conjugation of the 3′-bio-PO-ODN to the NLA-OX26 vector, the major clearance organ was the liver.

None of the studies on biotinylated oligonucleotides cited hereinabove discloses any complex comprising a biotin-binding molecule bound to a biotinylated double-stranded polynucleotide or to any polynucleotide encoding a functional polypeptide or transcriptional unit. In addition, no complex comprising an oligonucleotide or polynucleotide molecule covalently coupled to more than one biotin moiety was disclosed in any of the above cited art.

Systems for transfer of polynucleotides into cells by non-covalent complexes of polynucleotides with cations, such as polylysine, in which the cation rather than the polynucleotide is linked to a carrier by the binding of biotin to a biotin-binding protein, also have been described. For instance, Wagner, E., Zatloukal, K., Cotton, M., Kirlappos, H., Mechtler, K., Curiel, D. T., Birnstiel, M. L., Proc. Natl. Acad. Sci. U.S.A. 89:6099-6103 (1992), discloses that coupling of adenovirus to transferrin-polylysine/DNA complexes, via a biotin-streptavidin bridge, greatly enhances receptor-mediated gene delivery and expression of transfected genes. The authors reported that such complexes yield virtually 100% transfection in tissue culture cell lines. In these methods adenovirus was coupled to polylysine, either enzymatically through the action of transglutaminase or biochemically by biotinylating adenovirus and streptavidinylating the polylysine moiety. Combination complexes containing DNA, adenovirus-polylysine, and transferrin-polylysine were shown to have the capacity to transfer the reporter gene into adenovirus-receptor- and/or transferrin-receptor-rich cells.

Transfer of polynucleotides into cells by similar non-covalent complexes of polynucleotides with polylysine linked to a carrier protein by a biotin-avidin bridge also have been described. For instance, Strydom, S., Van Jaarsveld, P., Van Helden, E., Ariatti, M. and Hawtrey, A., J. Drug. Target 1:165-174 (1993), discloses the transfer of DNA into cells through use of avidin-polylysine conjugates complexed to biotinylated transferrin and DNA. Poly-L-lysine 460 was covalently attached to the carbohydrate chains of avidin via periodate oxidation and NaBH₄ reduction to give avidin-pLys460. Following purification through Sephacryl S-300, the conjugate was reacted with biotinylated transferrin. The conjugate was shown to bind DNA strongly, giving stable complexes soluble in 0.15-0.2 M salt solutions. Gene transfer using avidin-pLys460-[bio-transferrin] and the luciferase plasmid pRSVL was accomplished with Hela cells, alpha T3 pituitary cells and a human melanoma cell line. Transfection was dependent on bio-transferrin and stimulated by the lysosomotropic agent chloroquine. The results were said to be consistent with a receptor-mediated endocytosis mechanism of DNA delivery for Hela cells and a combination of receptor and adsorptive endocytosis for the alpha T3 pituitary and melanoma T-5 cell lines.

Similarly, synthetic virus-like gene transfer systems with a streptavidin-biotin bridge linking an endosome-disruptive peptide to polylysine complexed with DNA have been used to study the influence of such peptides on gene transfer by receptor-mediated endocytosis. Plank, C., Oberhauser, B., Mechtler, K., Koch, C. and Wagner E., Biol. Chem. 269:12918-12924 (1994). The process by which viruses destabilize endosomal membranes in an acidification-dependent manner was mimicked with synthetic peptides that are able to disrupt liposomes, erythrocytes, or endosomes of cultured cells. When such peptides were incorporated into DNA complexes that utilize a receptor-mediated endocytosis pathway for uptake into cultured cells, either by ionic interaction with positively charged polylysine-DNA complexes or by a streptavidin-biotin bridge, a strong correlation between pH-specific erythrocyte disruption activity and gene transfer was observed. A high-level expression of luciferase or interleukin-2 was obtained with optimized gene transfer complexes in human melanoma cells and several cell lines.

More recently, Madon, J. and Blum, H. E., Hepatology 24:474-481 (1996), discloses receptor-mediated delivery of hepatitis B virus DNA and antisense oligodeoxynucleotides to avian liver cells using a system similar to that of Wagner et al., supra, containing streptavidin, but without biotinylation of the adenovirus in the complex. The paper discloses a receptor-mediated delivery system for DNA and oligodeoxynucleotides (ODNs) to avian liver cells, using complexes of nonmodified human adenovirus particles and a protein conjugate consisting of N-acetyl-glucosamine-modified bovine serum albumin, streptavidin, and Poly-L-lysine. The method of protein-conjugate preparation and purification was reported to yield highly stable complexes with high DNA delivery efficiency for constructs expressing the lacZ gene, hepatitis B virus (HBV) DNA, and ODNs.

Polynucleotide Complexes in Applications other than Delivery to Viable Cells

Various complexes of linear oligo- and/or polynucleotides coupled together at both ends have been disclosed for various purposes other than delivery of the complexes into viable cells. For instance, Elghanian, R., Storhoff, J. J., Mucic, R. C., Letsinger, R. L., Mirkin, C. A., Science 277:1078-1081 (1997), discloses detection of polynucleotides based on the distance-dependent optical properties of gold nanoparticles held together by hybridization of complementary polynucleotides. A highly selective, calorimetric polynucleotide detection method based on mercaptoalkyloligonucleotide-modified gold nanoparticle probes is described. Introduction of a single-stranded target oligonucleotide (30 bases) into a solution containing the appropriate probes resulted in the formation of a polymeric network of nanoparticles with a concomitant red-to-pinkish/purple color change. Hybridization was facilitated by freezing and thawing of the solutions, and the denaturation of these hybrid materials showed transition temperatures over a narrow range that allowed differentiation of a variety of imperfect targets. Transfer of the hybridization mixture to a reverse-phase silica plate resulted in a blue color upon drying that could be detected visually. However, the disclosed oligonucleotides were not linked to each other by interaction of ligands specifically binding to a ligand-binding molecule; and, in any event, there is no mention of using the disclosed gold nanoparticles, either separately or in the described networks, for delivery of polynucleotides to viable cells.

European Patent Application EP 0 798388A1, published Oct. 1, 1997, discloses a method for detecting a gene, which method comprises reacting a double-stranded gene, which has been amplified with the use of gene fragments having an antigen or an antibody bonded thereto, with particles having an antibody or an antigen recognizing said antigen or antibody bonded thereto, or particles having a substance specifically binding to said antigen or antibody bonded onto the surface thereof, and measuring the degree of agglutination of the particles to thereby detect the target gene. Also disclosed is a method for detecting a mutation in a gene by using the above method. This disclosure states that in this method, “it is possible to utilize the specific bond between biotin/avidin or streptavidin, ligand/receptor, sugar chain/lectin or enzyme/inhibitor. In such a case, one is bonded to the primers while another is bonded to the particles.” Page 5, lines 24-26. The invention method is exemplified by dsDNA fragments, synthesized by PCR using 3′-biotinylated oligonucleotide primers, and latex beads coated with anti-biotin antibody. However, there is no mention in this disclosure of using any composition for delivery of polynucleotides to viable cells.

Despite the development of a wide array of compositions and methods for delivery of polynucleotides into mammalian and other similar eukaryotic cells, such as those described above and many others not cited herein, there remains a need for polynucleotide delivery approaches which provide greater ease of manufacturing, including convenient control of the stoichiometry of components in the delivery composition and facile assembly of diverse oligonucleotides or polynucleotides into a single complex for concomitant delivery into the same cell, as well as efficient uptake and high level expression of encoded genes by targeted cells. Notably, none of the above cited work appears to disclose delivery of polynucleotides into cells using a complex in which each polynucleotide molecule is specifically linked to other polynucleotide molecules by bridges comprised of ligand-binding molecules specifically bound to ligands coupled to the polynucleotides. More particularly, the prior art does not appear to have contemplated delivery of polynucleotides linked to each other at multiple points on each molecule, for instance, linear double-stranded DNA molecules linked at both ends, using bridges comprised of ligand-binding molecules having multiple ligand-binding sites bound to ligand moieties coupled at multiple points on each polynucleotide molecule, thereby forming a three-dimensional polynucleotide complex.

### SUMMARY OF THE INVENTION

One object of the present invention is to provide a compositions for delivery of polynucleotides into viable cells, which compositions comprise complexes of polynucleotides held together by ligand-binding molecules specifically bound to ligand moieties that are covalently coupled to the polynucleotides. It is a further object of the invention to provide such complexes which, compared to known polynucleotide delivery systems, provide more convenient self-assembly and better control of the stoichiometry of components in the composition. Another object of this invention is to provide a method for convenient incorporation of diverse oligonucleotides or polynucleotides into a single complex for concomitant delivery into the same cell. It is also an object of the invention to provide compositions and methods for efficient uptake of polynucleotides by targeted cells, as well as high level expression of genes encoded by delivered polynucleotides. These and other objects are provided by compositions and methods of the present invention.

The present invention is based in part on the discovery that compositions comprising particular complexes of polynucleotides, held together by ligands and ligand-binding molecules, provide enhanced uptake and expression of the complexed polynucleotides in viable cells, particularly in mammalian cells, compared to polynucleotides not in such complexes. Complexes of the invention generally comprise polynucleotide molecules covalently coupled to ligand moieties which are specifically bound to ligand-binding molecules. Generally, each of the polynucleotide molecules in these complexes is covalently coupled to at least one of the ligand moieties, and each of the ligand-binding molecules comprises more than one of the ligand-binding sites. In preferred compositions of the invention, the number of polynucleotide molecules in the complexes equals most or all of the ligand-binding sites of the ligand-binding molecules in the complexes. In such preferred compositions, polynucleotide molecules in the complexes also comprise most or all of the polynucleotide molecules in the composition.

For example, in a particularly preferred embodiment described in the Examples, below, a composition of the invention comprises polynucleotides covalently coupled to biotin complexed with a biotin-binding protein, particularly a protein known as “neutral” avidin (“neutravidin”). In the simplest form of this exemplary composition, most or all of the biotinylated polynucleotide molecules and most or all of the avidin molecules are bound together in individual “unit” complexes (some forms of which are also called “GeneGrids” hereinbelow). Each such unit complex comprises a single molecule of neutravidin having each of its four biotin-binding sites bound to a biotin moiety, where each bound biotin moiety is covalently coupled to an end of a different polynucleotide molecule. Thus, each of these exemplary unit complexes contains four polynucleotide molecules bound together by a neutravidin molecule that is specifically bound to biotin moieties on the polynucleotide ends.

Accordingly, one aspect of the present invention, in general terms, relates to a composition comprising complexes which comprise polynucleotide molecules covalently coupled to ligand moieties, these ligand moieties being specifically bound to ligand-binding sites of ligand-binding molecules in the complexes. In this composition, each of the polynucleotide molecules is covalently coupled to at least one of the ligand moieties, and each of the ligand-binding molecules comprises more than one of the ligand-binding sites specific for the ligand moieties. Further; in preferred embodiments of this composition, most or all of the ligand-binding sites of the ligand-binding molecules in the complexes are complexed with a ligand moiety of a polynucleotide molecule, and most or all of the polynucleotide molecules in the composition are bound to ligand-binding molecules in the complexes. More in particular, the number of the polynucleotide molecules in the complexes is equal to at least about 50% of the total number of ligand-binding sites of all of ligand-binding molecules in the complexes, and it is also greater than about 50% of all polynucleotide molecules in the composition.

Unit complexes of the invention may be used to produce a more extensive, three-dimensional polynucleotide complex or “network of the invention, in which each polynucleotide molecule is coupled to more than one ligand moiety, thereby allowing each polynucleotide molecule in these complexes to be bound by ligand moieties to more than one ligand binding molecule. For instance, in a particularly preferred composition of the invention comprising polynucleotide networks, linear double-stranded DNA molecules having one biotin moiety coupled to the 5′ end of each DNA strand are bound by those two biotin moieties to two neutravidin molecules, with one neutravidin molecule being bound at each end of each double-stranded DNA molecule. Further, it is preferred that most neutravidin molecules in this particular exemplary composition are specifically bound to biotin moieties of four DNA molecules in the complexes, thereby forming a three-dimensional polynucleotide complex or network of the invention. Compositions of the invention comprising polynucleotide networks are particularly preferred for delivery of polynucleotides into viable mammalian cells, particularly networks of linear double-stranded DNA molecules having a biotin moiety coupled to the 5′ end of each DNA strand and bound by those biotin moieties to two neutravidin molecules.

Another aspect of this invention therefore relates to a composition of the invention, as above, in which the polynucleotide molecules comprise linear DNA molecules which are at least partially double-stranded, and each of these DNA molecules is covalently coupled to two ligand moieties, one of the ligand moieties being covalently coupled to the 5′ end of each strand of the DNA molecules. In preferred embodiments of this composition, the number of polynucleotide molecules specifically bound to ligand-binding sites in the complexes is greater than about 80% of all polynucleotide molecules in the composition. Also preferred are such compositions in which each of the ligand-binding molecules comprises four of the ligand-binding sites, particularly such compositions in which the ligand moieties comprise biotin moieties and the ligand-binding sites comprise biotin-binding sites.

In general, compositions of the invention may be prepared by contacting suitable ligand-binding molecules with a sample of polynucleotide molecules covalently coupled to ligand moieties under conditions such that the ligand-binding sites on the ligand binding molecules bind specifically to the ligand moieties which are covalently coupled to the polynucleotide molecules. Suitable ligand-binding molecules comprise multiple ligand-binding sites which bind specifically to the ligand moieties covalently coupled to the polynucleotide molecules. “Ligand moiety” in the context of the present invention means a ligand or a derivative thereof coupled to a polynucleotide, where the derivative binds specifically to a ligand binding site of a ligand binding molecule that binds specifically to the original (free) ligand before coupling to a polynucleotide, with substantially the same binding affinity as the original ligand. “Ligand-binding molecule” in the present context includes a polypeptide or protein or an analog of thereof. For instance, various non-polypeptide analogs of antibodies and other ligand-binding molecules are known in the art. In particular, a ligand-binding “molecule” in the present context includes proteins comprising a single polypeptide chain or multiple polypeptide chains, or “subunits,” which may be the same or different, whether covalently linked (different chains in an antibody, for instance) or non-covalently associated (for instance, the four monomeric subunits associated in a biotin-binding protein, such as avidin, which are not covalently coupled in the natural protein).

For instance, suitable pairs of ligand moieties and cognizant ligand-binding molecules include an antigen moiety and an antibody or fragment thereof which specifically binds to the antigen moiety, an oligosaccharide moiety and a lectin-binding protein or fragment thereof which specifically binds to the oligosaccharide moiety, and an enzyme inhibitor moiety and an enzyme or fragment thereof which specifically binds to the enzyme inhibitor moiety. Particularly preferred are a biotin moiety and a biotin-binding protein or fragment thereof which specifically binds to a biotin moiety. The biotin-binding moiety may be avidin or an avidin analogue known in the art, such as streptavidin, Neutra-lite avidin, neutral avidin, Lite-avidin, and succinylated avidin. Neutravidin as described in the Examples below is particularly preferred in compositions of this invention for delivery of polynucleotides into mammalian cells. Other suitable ligands include a heme moiety, a borate moiety or a “polypeptide nucleic acid (PNA) clamp,” for which suitable ligand-binding molecules are known in the art.

Each polynucleotide molecule in a unit complex of the invention may be covalently coupled to more than one ligand moiety, and multiple ligand moieties on each polynucleotide molecule may be the same or different (e.g., two different biotin analogs recognized by avidin) and may be recognized by the same or different ligand-binding moieties (e.g., biotin, recognized by avidin, and an antigenic determinant, recognized by an antigen-binding site of an antibody). Generally, unit complexes with multiple ligand moieties on each polynucleotide molecule may be prepared by mixing polynucleotides covalently coupled to multiple ligand binding moieties with ligand-binding molecules having multiple binding sites that bind specifically to at least one ligand moiety on the polynucleotides. When each polynucleotide molecule is coupled to multiple ligand moieties that bind to different ligand-binding molecules (e.g., biotin and a carbohydrate moiety), unit complexes may be produced by contacting the polynucleotide molecules with a single species of ligand-binding molecule (e.g., avidin or a lectin-binding molecule).

To produce unit complexes of the invention, for instance, advantageously each polynucleotide molecule in the sample is coupled to a single ligand moiety which is preferably coupled to one end of the polynucleotide molecule. By “coupled to one end” is meant that the ligand moiety is couple to the last nucleotide on the indicated end or on a nucleotide that is near the last nucleotide on the indicated end. The ligand moieties are covalently coupled to the polynucleotide molecules either directly or by a linker moiety, using any conventional chemistry known in the art of making nucleotide derivatives.

Also, for unit complexes, advantageously the total number of ligand-binding sites of all ligand-binding molecules contacted with the polynucleotide sample is less than the number of ligand moieties coupled to the polynucleotide molecules in the sample. In other words, unit complexes of the invention are produced when a molar excess of singly biotinylated polynucleotide molecules, for example, is contacted with biotin-binding molecules, where the molar excess is calculated on the basis of the total number of biotin moieties on polynucleotides in the polynucleotide sample and the total number of biotin-binding sites in the biotin-binding molecules contacted with that sample. In contrast to these conditions, a previous report of enhancement of cellular uptake of a biotinylated antisense oligonucleotide mediated by a biotin-binding protein used conditions in which a vast molar excess of the biotin-binding protein was contacted with a sample of biotinylated oligonucleotides. See Pardridge, W. M. and Boado, R. J., 1991, supra, at page 32. These latter conditions would necessarily produce compositions comprising complexes that would be predominantly bimolecular, in which a single biotinylated oligonucleotide would be bound to a single molecule of biotin-binding protein, as well as a substantial amount of free biotin-binding protein not bound to any oligonucleotide, most likely more free biotin-binding protein than such protein in the bimolecular complexes.

Accordingly, another aspect of this invention relates to a method of making a composition of the invention comprising: contacting ligand-binding molecules with a sample of suitable polynucleotide molecules under conditions such that ligand-binding sites on the ligand binding molecules bind specifically to the ligand moieties which are covalently coupled to the polynucleotide molecules. In this method, preferably the total number of ligand-binding sites of all of ligand-binding molecules contacted with the polynucleotide sample is less than the number of ligand moieties coupled to the polynucleotide molecules in the sample. For instance, advantageously the total number of ligand-binding sites contacted with the sample is at least about ten times less than the number of ligand moieties coupled to the polynucleotide molecules in the sample.

Compositions of the invention may be enriched for unit complexes containing ligand-binding molecules with all ligand-binding sites bound to ligand moieties on polynucleotides by methods such as affinity chromatography, for instance, using a substrate coupled to an appropriate ligand moiety to remove ligand-binding molecules with any unoccupied ligand-binding site, or, where each polynucleotide is coupled to a single ligand moiety, using a substrate coupled to an appropriate ligand-binding molecule to remove polynucleotide molecules with an unbound ligand moiety. As illustrated in the Examples, for instance, the yield of unit complexes produced by the above method may be improved, after contacting the ligand-binding molecules with the polynucleotide sample, by removing some of the polynucleotide molecules covalently coupled to ligand moieties that are not bound to the ligand-binding sites of ligand-binding molecules in the sample. In this method, polynucleotide molecules covalently coupled to ligand moieties that are not bound to ligand-binding sites in complexes are removed from a sample by contacting the sample with a solid support coated with ligand-binding molecules specific for the ligand moiety of the polynucleotides, under conditions such that the ligand moieties of polynucleotides that are not bound to ligand-binding molecules in the complexes specifically bind to ligand-binding molecules on the solid support. Then the sample enriched for complexes is recovered by separating the complexes remaining in the sample from the solid support. Removal of unbound polynucleotides from compositions comprising complexes of the invention may be accomplished by various other means known in the art, including chromatographic methods based on differential size.

Compositions of the invention comprising more extensive complexes or networks, in which each polynucleotide molecule is coupled to more than one ligand moiety and is thereby bound to more than one ligand binding molecule, also may be produced simply by contacting suitable ligand-binding molecules with a sample of such polynucleotide molecules under conditions such that the ligand binding molecules bind specifically to the ligand moieties of the polynucleotide molecules. For example, a particularly preferred composition of the invention comprises polynucleotide networks of linear double-stranded DNA molecules having one biotin moiety coupled to the 5′ end of each DNA strand in each double-stranded DNA molecule, and each double-stranded DNA molecule in such networks is thereby bound to two biotin-binding molecules. Such a preferred composition may be produced by simply contacting biotin-binding molecules with these double-stranded DNA molecules.

However, the extent of network formation of such linear double-stranded DNA molecules coupled to two biotin moieties, upon contacting them with biotin-binding molecules, depends upon the ratio of the number of biotin moieties of such DNA molecules relative to the number of biotin-binding sites on the biotin-binding molecules contacted with these DNA molecules. As explained in the Examples, below, it is apparent that contacting a vast excess of biotin-binding molecules with a sample of such DNA molecules would be expected to produce predominantly biomolecular complexes of one DNA molecule with one biotin moiety bound to one biotin-binding molecule. It is also apparent that contacting a sample of biotin-binding molecules with a vast excess of such double-stranded, doubly biotinylated DNA molecules would be expected to produce predominantly complexes of one biotin-binding molecule having each biotin-binding site bound to a biotin moiety of a different DNA molecule. As further shown in the Examples, at certain ratios of such DNA molecules to biotin-binding molecules, an estimated 50% of the input double-stranded, doubly biotinylated DNA molecules formed complexes with biotin-binding molecules.

Compositions comprising substantially more than about 50% of the above described double-stranded, doubly biotinylated DNA molecules in complexes with biotin-binding molecules have not been observed when a sample of such DNA molecules is simply contacted with any amount of biotin-binding molecules at one time (that is, when a single sample of such DNA molecules is contacted with a single amount of biotin-binding molecules).

The yield of networked DNA may be increased, for instance, by incrementally adding small amounts of avidin to an excess of polynucleotide molecules, thereby effectively “seeding” the mixture with unit complexes which are then extended and linked together by additional avidin molecules to produce networked DNA. Alternatively, polynucleotide networks of the invention may be assembled by mixing purified unit complexes, which comprise ligand-binding molecules saturated with polynucleotides covalently coupled to multiple ligand moieties, with additional free ligand-binding molecules which then cross-link free ligand moieties on polynucleotides in different unit complexes. More in particular, compositions comprising at least about 80% of such double-stranded, doubly biotinylated DNA molecules in complexes with biotin-binding molecules may be obtained by a seeding procedure in which small amounts of biotin-binding molecules are successively contacted with a sample of such DNA molecules, such that a relatively low ratio of biotin-binding molecules to such DNA molecules (for instance, less than one biotin-binding molecule to more than one hundred of such DNA molecules) is maintained while contacting each successive amount of biotin-binding molecules with the DNA sample. In this method, preferably the total number of ligand-binding sites in each of the small amounts contacted with the DNA sample is at least about 100 times less than the number of ligand moieties coupled to DNA molecules in the sample. Accordingly, compositions of the invention, as described herein, include compositions in which the number of polynucleotide molecules specifically bound to ligand-binding sites in complexes of the invention is greater than about 50%, preferably at least about 60% to about 80%, more preferably at least about 80% to about 90%, still more preferably greater than about 90%, for instance, about 95%, 97% or 99%, of the total number of ligand-binding sites of all of ligand-binding molecules in the complexes in the composition. Similarly, compositions of the invention include compositions in which the number of polynucleotide molecules specifically bound to ligand-binding sites in complexes of the invention is at least about 50%, preferably at least about 60% to about 80%, more preferably at least about 80% to about 90%, still more preferably greater than about 90%, for instance, about 95%, 97% or 99%, of all polynucleotide molecules in the composition.

Compositions comprising double-stranded, doubly biotinylated DNA networks also may be efficiently produced by first producing and purifying unit complexes of the invention comprising such DNA molecules, followed by contacting such unit complexes with additional biotin-binding molecules. Compositions comprising polynucleotide networks of the invention also may be produced by other methods described herein or which would be readily apparent to one of ordinary skill on reading the present disclosure. For instance, unit complexes of double-stranded polynucleotides coupled to multiple ligand moieties (e.g., a first and second ligand moiety, which may bind to the same or different ligand-binding molecules) may be produced by initially saturating a first ligand-binding protein with multiple ligand-binding sites for a first ligand moiety with a first single-stranded polynucleotide coupled to a first ligand moiety, and then annealing to the first polynucleotide a second single-stranded polynucleotide coupled to a second ligand moiety. In this approach, the complex of first ligand moiety and first ligand-binding molecule must be stable to the conditions, such as heating, which are used for annealing of complementary polynucleotide strands. Alternatively, networks may be produced by adding ligand moieties to polynucleotides in unit complexes, by conventional chemical methods, followed by addition of ligand-binding molecules which specifically bind to the added ligand moieties.

A previous report of biotinylated double-stranded DNA molecules complexed with anti-biotin antibodies did not disclose formation of such complexes using a seeding procedure of the present invention, in which small amounts of biotin-binding molecules are successively contacted with a sample of such DNA molecules, such that a relatively low ratio of biotin-binding molecules to such DNA molecules (for instance, less than one biotin-binding molecule to more than one hundred of such DNA molecules) is maintained while contacting each successive amount of biotin-binding molecules with the DNA sample. See European Patent Application EP 0 798388A1, published Oct. 1, 1997. Accordingly, the conditions disclosed in the report would not be expected to produce compositions in which a high percentage of all double-stranded DNA molecules with in complexes with biotin-binding antibodies bound to each biotin-biotin binding site of said antibodies. FIG. 2 of this report shows that most of the particles in an exemplary composition, to which the biotin-binding antibodies are bonded, remain unagglutinated or agglutinated only in particle dimers or trimers, after contact with the biotinylated double-stranded DNA molecules. This is consistent with most of the biotin-binding sites on those particles, each of which comprises multiple biotin-binding sites of many antibody molecules, not being bound to biotin moieties on DNA molecules which are bound to two antibody molecules (on two particles). In any event, there is no disclosure in this application of any complexes consisting essentially of polynucleotides bound to each other only by binding of a ligand to a ligand-binding molecule, that is, complexes in which the ligand-binding molecule is not covalently coupled to a particle, such as the disclosed latex beads, bridging two or more ligand-binding molecules. Further, presumably the 0.8 micron diameter latex particles used in the disclosed exemplary composition would interfere with polynucleotide uptake by viable cells, if such delivery were attempted with the disclosed exemplary composition.

In general, the polynucleotide of a complex of the invention may be single stranded (DNA, RNA or a polynucleotide analog), or partially or wholly double-stranded, where the two strands are held together by hydrogen bonding of complementary nucleotide sequences in the two strands (i.e., by annealing or hybridization, the latter involving two different polynucleotides in the double-stranded region, e.g., DNA and RNA). “Polynucleotide” in the present context includes oligonucleotides and polynucleotides as well as analogs of either, such as “polypeptide nucleic acids (PNAs)” or derivatives or nucleic acids with sulfur replacing the natural phosphorus in the subunit coupling chemistry, all of which are known in the art. Further, the polynucleotide “molecule” in complexes of the invention may be circular or linear, and, in either case, may be at least partially double-stranded, that is, it may comprise one or more single-stranded segments linked by annealing of overlapping complementary portions of different polynucleotide strands. Further, polynucleotide molecules in compositions of the invention may encode a polypeptide, including an peptide or oligopeptide, or a complete transcriptional unit comprising a sequence encoding a polypeptide. Polynucleotides in compositions of the invention also include “antisense” oligonucleotides, that is, a polynucleotide molecule encoding a sequence of at least ten nucleotides which is complementary to at least ten nucleotides of a nucleotide sequence encoding some portion of a transcriptional unit, encoding either a regulatory sequence or an amino acid sequence of a polypeptide.

Other aspects of the invention relate to a method of delivering polynucleotide molecules to a viable cell comprising contacting a composition of the invention, particularly a composition comprising networks of double-stranded DNA, with the viable cell. In a preferred embodiment of this method, the complexes in the composition contacted with the viable cell are contained in liposomes, as exemplified below. In general, complexes in compositions of the invention used for delivery of polynucleotides to a viable cell also optionally further comprise at least one component which enhances uptake of the polynucleotides in the complexes, such as a ligand for a receptor or a nuclear transport peptide, and the like. These and other aspects of the invention are further described below.

### BRIEF DESCRIPTION OF THE DRAWINGS

FIG. 1: Crystal structure of a biotin-avidin complex.

Arrows indicate biotin molecules. Notice the 2-fold symmetry of avidin tetramers. The coordinates were obtained from the Brookhaven Protein Database (PDB access code: 1 AVD).

FIG. 2: Principles of BANG by photobiotinylation or by PCR

Panel A: BANG by photobiotinylation; panel B: BANG by PCR.

FIG. 3: Schematic illustration of the GRASP purification procedure.FIG. 4: Strategies of formation and purification of biotin-avidin networked gene system (BANG).

FIG. 5: Indirect-labeled immunofluorescence (IF) pictures of Hela cells after kDNA transfection.

Hela cells transfected with photobiotinylized kDNA (left two panels) or without (right panel, mock) were faxed and permeablized as described in the Materials and Methods. Avidin conjugated FITC were used to visualize the photobiotinylized kDNA. In the mock transfected cells (right panel), only background fluorescence was observed. In the kDNA-transfected cells (left two panels), most labeling appeared around the peripheral region of the nucleus.

FIG. 6: Formation of BANG using photobiotinylation.

Plasmid DNA (pTS-Luc, 1 μg/μl) was photobiotinylated before avidin was added to form BANG DNA (Panel A, arrow). The same DNA samples were used on a southern blot probed with avidin-conjugated alkaline phosphate (panel B). Controls were performed as indicated on the pictures.

FIG. 7: Luciferase activities of BANG generated by photobiotinylation.

DNA were photobiotinylated before crosslinking by avidin. The same mount of each DNA was transfected using lipofectamine in the supercoiled, linear and BANG DNA samples. No DNA was used in the mock samples. Error bars were calculated from duplicate transfections.

FIG. 8: Examination of biotinylated PCR products.

Biotinylated DNA from PCR reactions were purified before incubating in a avidin-coated multiple-well titration plate (MTP) for 60 min. on a shaking platform at room temperature (lanes 5 and 7: with incubation: lanes 6 and 8: without incubation). Non-biotinylated calf thymus DNA were used as negative controls (lanes 1 through 4). Lane M is 1 kb ladder for DNA molecular weight references. Arrow: PCR monomer (2.58 kb).

FIG. 9: Formation of BANG by PCR.

PCR was performed using pGL3 as template with biotinylated primers (see Materials and Methods). Avidin was added at various amount. Reactions were incubated at room temperature for 2 hours before loading onto a 0.8% agarose gel. The gel was stained with EtBr before documentation. Lane :M, 1 kb ladder. Lane 8 control PCR DNA without addition of avidin. Lane 2-7, avidin titration experiments.

FIG. 10: BANG formation by PCR and seeding procedure.

Avidin was first incubated for 2 hours at room temperature with biotinylated DNA (400 ng) in a very limited amount for seed formation (lanes 1 and 2: 0.52 ng of avidin; lanes 3 and 4: 3.1 ng of avidin). 31 ng of avidin was then added as a second step (lane 2 and 4). Seeding controls (without second step growth) are in lanes 1 and 3. Negative controls are in lanes 5 (biotinylated monomers without avidin) and lane 6 (NBD: non-biotinylated DNA with avidin). Lane M: 1 kb DNA ladder.

FIG. 11: Transmission electron microscope pictures of BANG DNA.

Biotin-labeled DNA molecules (size: 2.58 kb) were obtained by PCR and prepared for EM as described in Materials and Methods. Panel A is the control monomer DNA where no avidin was added (×45000), Panels B and C are BANG DNA under the TEM at different magnification (B: ×75000; C: ×125000).

FIG. 12: Scanning electron microscope pictures of BANG DNA.

BANG DNA were formed as described. Clockwise from the top left are panels A, B, C and D. Panel A and D are the same BANG DNA at different magnifications: ×8000 and ×45000, respectively. Panel B is a pure avidin molecule. Panel C is a dimer DNA.

FIG. 13: Effect of temperature on BANG formation.

BANG DNA were formed without the seeding procedure as described before (FIG. 9, lane 5), except that the incubation temperatures were different (as indicated on the picture).

FIG. 14: GeneGrid on a two-dimension agarose gel after the GRASP procedure.

Gene,Grid was formed and purified as described in Materials and Methods. Panel A: 2-D gel; Panel B: schematic illustration of the expected results.

FIG. 15: Strategies of in vitro characterization of BANG.

FIG. 16: Restriction digestion of GeneGrid DNA.

GeneGrid DNA were generated without GRASP purification. Restriction digestion were performed at 37° C. for 2 hours before inactivated at 65° C. for 10 min. Panel A: schematic illustration of the restriction digestion. Panel B: electrophoresis results of the restriction digestion of GeneGrid DNA. G: GeneGrid DNA. C: Control DNA (monomer). Lanes I and 2: Xho. I cut; Lanes 3 and 4: Hind III cut; Lanes 5 and 6: Xba I cut; Lanes 7 and 8: controls without any restriction enzymes. Lane 9 and 10: controls for 65° C. treatment. Lane 11: monomer control; Lane M: 1 kb DNA ladder.

FIG. 17: Temperature effect on stability of BANG DNA

Equal amounts of BANG DNA (800 ng) were presented in each reaction before the, were subjected to incubation at different temperature. Lanes 8, 9, and 10 were controls as indicated. Lanes 1 to 7 were BANG DNA incubated at a specific temperature as labeled. Lane M: 1 kb DNA ladder.

FIG. 18: Stability of the BANG system in the presence of proteinase K.

BANG DNA were formed without the seeding procedure as described in Chapter 6. The reactions were then incubated with proteinase K (100 μg/ml) at 37° C. for 2 hours (lanes 2, 4, and 6) or without (lanes 1, 3, and 5). Lane 7 was the control (monomer). Lane M: I kb DNA ladder.

FIG. 19: Strategies of in vivo characterization of BANG.

FIG. 20: Cellular distribution of BANG DNA.

Hela cells were transfected with BANG DNA (panel B and C) or without any DNA (mock; panel A). Monoclonal anti-avidin antibodies were used as primary antibody and goat-anti-mouse FITC-conjugated antibodies were employed as secondary antibodies.

FIG. 21: IF pictures of luciferase expression in individual cells (part 1 of 3): controls.

PCR products of pGL3-control DNA (luciferase under control of CMV promoter) were used in this study. NIH 3 cells were transfected with monomer control DNA (C and D: monomers without biotinylation and without avidin; and F: monomers with biotinylation but without avidin), or without any DNA (mock transfection, A and B).

FIG. 22: IF pictures of luciferase expression in individual cells (part 2 of 3): BANG DNA.

Different fields of BANG DNA transfected cells. Phase contrast pictures: A, C, and E. Corresponding IF pictures: B, D, and F.

FIG. 23: IF pictures of luciferase expression in individual cells (part 3 of 3): BANG DNA over-expression.

One field of cells transfected with BANG DNA is illustrated here. Phase contrast: A. IF pictures: B and C. Panel C was taken using one hundredth exposure time as that of B.

FIG. 24: Transfection efficiency (positive percentage) obtained by flowcytometry.

Luciferase DNA (pGL3-control) were used in PCR reactions. Hela cells were transfected with no DNA (mock), PCR monomer DNA (Monomer transfected) or BANG DNA (BANG transfected), as described in Materials and Methods. Cells were fixed and labeled with anti-luciferase antibody 48 hours after transfection. FITC conjugated secondary antibody was used for cytometer analysis. Total cells were counted along with the positive cells, which were identified as their FITC signals above those of mock transfected. Positive cell numbers were then divided by the total cell numbers to obtain positive percentage, which indicated transfection efficiencies.

FIG. 25: Cytotoxicity study of BANG after transfection.

HL60 cells were transfected with BANG DNA, monomer DNA or mock (luciferase ). Cell numbers were counted at specified time in triplicates.

FIG. 26: Luciferase activity of Hela cells transfected with BANG DNA

BANG DNA were transfected to Hela cells, along with controls (monomer DNA. supercoiled plasmid DNA and mock). Luciferase activities were measured in terms of relative light unit (RLU), and were normalized to total cell numbers of each sample (using total pg of DNA). The amount of the plasmid DNA, pGL3-control, was adjusted to the same mole amount as the monomer DNA.

FIG. 27: Luciferase activity of NIH3T3 cells transfected with BANG DNA.

NIH3T3 cells were first seeded in a 35 mm culture dish containing one coverslip. BANG DNA and various control DNA were delivered by lipofectamine. Cells grown on the coverslip were fixed ad stained with anti-luciferase antibodies. The transfection efficiencies were obtained and were used to normalize the luciferase assay results. The rest cells were harvested for luciferase assays. nb+na: no biotin monomer with no avidin incubation: nb+a: biotinylated monomer with no avidin incubation: nb+a: non-biotinylated with avidin incubation.

FIG. 28: GFP expression histogram in Hela cell.

### DETAILED DESCRIPTION OF THE INVENTION

In one aspect, the invention relates to a composition comprising complexes which comprise polynucleotide molecules covalently coupled to ligand moieties, these ligand moieties being specifically bound to ligand-binding sites of ligand-binding molecules in the complexes. Polynucleotide molecules in the complexes comprise nucleotides such as deoxyribonucleotides, ribonucleotides, analogs of deoxyribonucleotides, and analogs of ribonucleotides, such analogs being known and available in the art. For instance, polynucleotides of the invention complexes include “peptide nucleic acids (PNAs),” DNA analogs containing neutral amide backbone linkages, which are stable to degradation by enzymes and hybridize to complementary sequences with higher affinity than analogous DNA oligomers. See, for instance, Corey, D. R., Trends Biotechnol 15:224-229 (1997).

As noted above, polynucleotide molecules in compositions of the invention may encode a polypeptide, including an peptide or oligopeptide, or a complete transcriptional unit comprising a sequence encoding a polypeptide. For instance, polynucleotides in compositions of the invention include linear PCR-amplified DNA fragments, or circular DNA molecules, in constructs designed for nuclear expression or cytoplasmic expression. See, for example, Li, S., Brisson, M., He, Y. and Huang, L., Gene Ther 4:449-454 (1997).

Ligand-binding molecules suitable for the present invention, besides those listed elsewhere herein, include antibodies, particularly antibodies of the IgM class which are multimeric and therefor have multiple binding sites, which specifically bind to a suitable ligand moiety.

As noted above, the ligand moieties of the invention is covalently coupled to the polynucleotide molecule either directly or by a linker moiety, using any conventional chemistry known in the art of making nucleotide derivatives. For instance, U.S. Pat. No. 5,585,481 to Arnold et al., discloses linking reagents for nucleotide probes which may be used in the present invention.

When the ligand moiety to be used is biotin or a derivative thereof, various means known in the art may be used to covalently couple that moiety to a polynucleotide. For instance, photobiotinylation of DNA as well as incorporation of biotin via PCR amplification of DNA using biotinylated oligonucleotide primers are described in the Examples below. In addition, use of T4 kinase for coupling of biotin to a polynucleotide is known to provided a simple, fast and efficient method of preparing 5′ biotin-labeled oligonucleotides. See, for instance, Harper, J. W., Lee, G. L. C. and Logsdon, N. Anal. Biochem. 205:36 (1992). Biotinylation of DNA, by nick-translation of double-stranded DNA using biotinylated dUTP, for example, may also be used in the practice of the present invention. See, for instance, Shimkus, M. et al., Proc. Natl. Acad. Sci. USA 82:2593-2597 (1985). U.S. Pat. No. 5,506,121, to Skerra; et al., discloses fusion peptides with binding activity for streptavidin which may be used as a biotin moiety of the invention.

A variety of biotin-binding molecules suitable for the present invention are known in the art. See, for instance, Green, N. M. Avidin and streptavidin. Methods Enzymol 184:51-67 (1990). Neutral avidin, which is avidin from which naturally occurring carbohydrate modifications have been removed, is particularly preferred for delivering polynucleotides into mammalian cells, as described in the Examples below. See, for instance, Hiller, Y., Gershoni, J. M., Bayer, E. A. and Wilchek, M. Biochem J 248:167-171 (1987). Minimized avidin fragments are known that bind biotin and these or similar fragment of neutral avidin or a related biotin-binding protein also may be used in the invention. See, for instance, Hiller, Y., Bayer, E. A. and Wilchek, M., Biochem J 278:573-585 (1991). As noted above, other avidin analogues suitable for the invention are also known, including streptavidin, Neutra-lite avidin, avidin, Lite-avidin, and succinylated avidin. See, for instance, Kang, Y. S., Saito, Y. and Pardridge, W. M., J. Drug Target 3:159-165 (1995).

In addition, analogs of avidin exhibiting reversibility of biotin-binding, produced by selective modification of tyrosine in avidin, have been described and also may be used in the present invention. See Morag, E., Bayer, E. A. and Wilchek, M., Biochem J 316:193-199 (1996). Chemically modified forms of avidin and streptavidin (termed nitro-avidin and nitro-streptavidin, respectively), in which the binding-site tyrosine was nitrated, also exhibit an interaction with biotin can be reversed under relatively mild conditions. Morag, E., Bayer, E. A., and Wilchek, M., Anal Biochem 243:257-263 (1996). Immobilized avidin or avidin analogs, particularly nitro-avidin and nitro-streptavidin, also may be used as affinity matrices for purification of polynucleotide complexes of the invention, by removing biotinylated polynucleotides in a sample which not bound to a biotin-binding protein in a complex of the invention.

Anti-biotin antibodies which are suitable for the present invention when a biotin moiety is coupled to a polynucleotide are also known. See, for example, Kohen, F. et al., Methods Enzymol 279:451-463 (1997); also see M. Hollinshead, J. Sanderson, D. J. Vaux, J. Histochem Cytochem. 45:1053 (1997).

Shin, S. U., et al., J. Immunol 158:4797-4804 (1997), describes functional and pharmacokinetic properties of antibody-avidin fusion proteins which may be used in the invention, for instance, to target the complexes to a surface antigen of a desired cell.

Optionally, the ligand-binding molecule in complexes of the invention may be modified chemically or enzymatically to improved characteristics such as rate of clearance from the circulatory system. For example, polyethylene glycol modification proteins is well known in the art, including such modification of streptavidin. See, for instance, Marshall, D. et al., Br. J. Cancer 73:565-572 (1996).

As noted above, complexes in compositions of the invention used for delivery of polynucleotides to a viable cell also optionally further comprise components which enhance uptake of the polynucleotides in the complexes, such as a ligand for a receptor or a nuclear transport peptide. For instance, such ligands include folate (Gottschalk, S. et al., Gene Ther 1:185-191(1994)), transferrin, low-density lipoprotein (LDL), polypeptide growth factors such as epidermal growth factor (EGF and related ligands) and fibroblast growth factors (FGFs), 6-mannose-phosphate, an integrin-binding peptide, or a toxin or fragment or subunit thereof which binds specifically to a surface receptor. Cell-binding peptides selected from random peptide-presenting phage libraries also may be used as optional cell-targeting components of polynucleotide complexes of the invention. Barry, M. A., et al., Nat Med 2:299-305(1996). Additional optional components also include surfactant proteins, viral particles or proteins or fragments thereof, or a chemical or a toxin which modifies lysosomal trafficking, such as lysotrophic amines or brefeldin, which are known in the art. For instance, influenza virus hemagglutinin HA-2 N-terminal fusogenic peptides may be used as a component to enhance cellular uptake of complexes of the invention. See, for instance, Wagner, E., et al., Proc. Natl. Acad. Sci. U.S.A. 89:7934-7938 (1992). Gottschalk, S., et al., Gene Ther 2:498-503 (1995), describes perfringolysin O, a member of the so-called sulfhydryl-activated family of membrane active bacterial proteins, which also can be used in complexes of the invention to enhance gene delivery and expression in mammalian cells using of polynucleotides in complexes of the present invention. A cationic peptide that binds to nucleic acids and permeabilizes bilayers also may be included in complexes of the invention. Wyman, T. B., et al., Biochemistry 36:3008-3017(1997).

Compositions of the invention used for delivery of polynucleotide complexes to cells also optionally comprise polycations such as diethylaminoethyl-dextran (DEAE-dextran) or poly-L-lysine (see, for instance, Ehrlich, M., Sarafyan, L. P., Myers, D. J., Biochim. Biophys. Acta 54:397-409 (1976)) or polyornithine, spermine, or polyarginine (see, for example, Farber, F. E., Melnick, J. L., and Butel, J. S., Biochim Biophys Acta 390:298-311 (1975)).

Compositions of the invention used for delivery of polynucleotide complexes to cells may be introduced into cells by any means known for introducing nucleic acids into cells, such as “biolistics” (Webster, R. G., et al., Vaccine 12:1495-1498 (1994)) or by electroporation (Nishi, T. et al., Cancer Res. 56:1050-1055 (1996)).

Polynucleotide compositions of the invention used for intramuscular injection of polynucleotides optionally are formulated as a complex with an agent, such as PVP, which provides sustained release of the polynucleotides in the composition. Alila, H., et al., Hum Gene Ther8:1785-1795 (1997).

Compositions and methods of the invention are useful for delivery of antisense oligonucleotides (Boado R. J. and Pardridge, W. M., Bioconjug Chem 5:406-410 (1994)) or polynucleotides encoding polypeptides, to viable cells in culture or in vivo, that is, cells which are part of a multicellular organism, including a mammal, particularly a human subject. For instance, compositions of the invention may be used for delivery of DNA vaccines, or for genomic targeting and genetic conversion in cancer therapy, as described, for instance, by Kmiec, E. B. in Semin. Oncol. 23:188-193(1996). The invention also is useful for transfection of mitochondria for gene therapy of mitochondrial DNA diseases. Seibel, P. et al., Nucleic Acids Res. 23:10-17(1995). The invention may also be used to deliver an enzymatically active RNA molecule (“ribozyme”) or a gene expressing a ribozyme, into a desired cell, to provide a desired enzymatic activity in that cell.

### EXAMPLES

NOTE: The full citation for each document cited in the following Examples is provided in the BIBLIOGRAPHY following the Examples.

All books, articles and patents cited in this specification are incorporated herein by reference in their entirety.

While the present invention has been described in connection with what is presently considered to be practical and preferred embodiments, it is understood that the present invention is not to be limited or restricted to the disclosed embodiments but, on the contrary, is intended to cover various modifications and equivalent arrangements included within the spirit and scope of the appended claims.

Thus, it is to be understood that variations in the described invention will be obvious to those skilled in the art without departing from the novel aspects of the present invention and such variations are intended to come within the scope of the claims below.

### Example 1

### From Topo II to K-DNA to Bang

DNA Crossovers and KDNA

Topo II is able to discern topologies of DNA by interacting preferentially with DNA crossovers. A natural substrate full of DNA crossovers is the kinetoplast DNA (kDNA).

KDNA is the mitochondrial DNA of trypanosomatid protozoa, which includes the African Trypanosoma brucei, the South American Trypanosoma Cruzi, and Crithidia fasciculata, a parasite of insects. It is a giant DNA network (˜10¹⁰ kDa) consisting of several thousand mini-circular DNA that are topologically interlocked (Marini et al. 1980). The minicircle DNA molecules are joined by a single interlock and each minicarcle is linked topologically to three neighboring minicircles (Chen et al. 1995). In a non-replication stage, all circular DNA in the network are covalently closed and fully relaxed (Rauch et al. 1993). The vast amount of DNA crossovers in the network make it a good substrate for topo II. As a matter of fact, kDNA is routinely used in vitro to detect topo II enzyme activity (decatenation assay).

Sensitizing Cells by KDNA Transfection

Topo II is one of the primary intracellular targets for a wide variety of clinically valuable anticancer agents (reviewed in Chen and Liu, 1994; Li, et al., 1994; Liu, 1989). The chemotherapeutic potentials of many of these agents are largely due to their abilities to trap and stabilize the covalent topo II-DNA intermediate complexes formed in topo II catalytic cycles (Osheroff, 1989; Robinson and Osheroff, 1990a). Since topo II preferentially binds to DNA crossovers and kDNA provides thousands of such crossovers, initially I proposed to use kDNA as a sensitizing reagent for topo II targeted cancer chemotherapy.

At least three questions must be answered before sensitization can be realized. First, could giant molecules such as kDNA be taken up by cells? Second, could kDNA be modified in such a way so that its sequences are altered but topology are left intact? In other words, could other genes be cloned into kDNA as part of a crosslinked network? Third, what kinds of effects would kDNA impose on normal cells? In the process of answering these questions, I realized that it was extremely difficult to modify the kDNA sequence without altering its topology. Cloning other genes into kDNA while keeping catenation intact was even more difficult to achieve. Instead of trying to modify kDNA directly, an alternative approach was applied—mimicking kDNA.

Mimicking KDNA Through Biotin-avidin T

Non-covalent Crosslinking

To mimic kDNA topology, circular DNA and topo II were initially used along with DNA condensing reagents in order to try to shift the catenation-decatenation equilibrium towards catenation. The result was, however, that the efficiency of catenation by this approach was extremely low. Since the DNA-crossovers were the sole reason to utilize kDNA as a sensitization reagent, and since recognition of crossovers by topo II was independent of torsional stress (Zechiedrich and Osheroff, 1990), an alternative approach of mimicking kDNA was applied—crosslinking DNA together.

In the process of finding such alternative approach, a whole new system was invented to crosslink DNA together. It turned out that this system was not just a way of linking DNA molecules together, but a novel way for gene over-expression and gene non-covalent cloning. This new system is called the “Biotin-Avidin Networked Gene” system (BANG system). The rest of this dissertation focuses on the formation, purification, in vitro characterization, and in vivo study of the BANG system after a background introduction to biotin, avidin and their interaction.

Background of Biotin

Biotin (cis-hexahydro-2-oxo-1H-thieno[3,4]imidazole-4-pentanotic acid, or C₁₀H₁₆N₂O₃S) is a naturally occurring small molecule (MW=244.31). Table 1 compiles the biotin chemical data adopted from the Merck Index (11th edition).

Biotin was first discovered to be a vitamin in 1927 when rats developed a malnutrition disease (dermatitis) after being fed with large quantities of egg white (Boas 1927). In 1940. B vitamin was chemically identified as biotin (György, et al., 1940, du Vigneaud et al. 1940). Soon it was found that the egg white avidin had an extra high affinity for biotin (György et al., 1941). In the fifties, biotin's role as a coenzyme in carbon dioxide transfer of carboxylating enzymes was recognized with the use of avidin as a biotinyl enzyme blocker (Lynen, et al., 1959; Wakil, et al., 1958). In the mid-1970s, biotinylation of membrane proteins for cytochemical application was developed (Heitzmann and Richards, 1974). Since then, biotin has been incorporated into many macromolecules without interference with their activity and has been widely used as a. general labeling reagent in cytochemistry, immunoassays, affinity. purification, and gene probes mainly due to its extremely high affinity to avidin (reviewed in Green, 1975; Wilchek and Bayer, 1990).

Background of Avidin

Avidin was first described as “toxic substances” in egg white by Bateman in 1916. (Bateman, 1916). In 1941, the extra high affinity of avidin for biotin was discovered (György et al., 1941). However, with no general biological function and no valuable clinical use, avidin research was inactive until the late fifties when avidin was used to block coenzyme function of biotin (Lynen, et al., 1959; Wakil, et al., 1958). Detailed protein chemistry studies were carried out by Green and others in the sixties and seventies (Green, 1966; Green and Melamed, 1966; Green, 1975). Table 2 shows chemical data of avidin-compiled from various sources (Bayer and Wilcheck, 1980; Green. 1975; Wilcheck and Bayer, 1990).

TABLE 1
Properties of Biotin

| Chemical Properties | Data |
|---|---|
| Element composition | 49.16°/aC. 19.65% O, |
| | 13.12°/aS. 11.47°/aN. |
| 6.60% H | |
| Molecular weight | 244.31 |
| Absorbance at 250 nm | 0.111 (1 mg/ml) |
| Absorbance at 280 nn | minimal |
| Isoelectric point | 3.5 |
| pH (0.01% aqueous solution) | 4.5 |
| Solubility in water at 25° C. | 0.22 mg/ml |
| Solubility in 95% alcohol | 0.80 mg/ml |

TABLE 2
Properties of Avidin

| Chemical Properties | Data |
|---|---|
| Subunits | 4 |
| Amino acid residues | 128 |
| Molecular weight (tetramer) | 67,000 Da-68,000 Da |
| Molecular weight (non-glycosylated) | 57 kDa total for four identical |
| monomer | |
| Absorbance at 282 nm | 1.54 (1 mg/ml) |
| Isoelectric pH | 10-10.5 |
| Oligosaccharide/subunit | 1 |
| Mannose/subunit | 4-5 |
| Biotin binding/subunit | 1 |

The amino acid sequence of avidin was first determined in 1971 (Delage and Huang, 1971). The gene was cloned from chicken in 1987 (Gope et al., 1987) Table3 lists the details of avidin protein primary sequences. Avidin is a highly specialized protein. Database searches revealed that no proteins identified so far had a significant similarity to avidin. Streptavidin, expressed in Streptomyces avidinii, is the only exception in that it has 33% of its residues identical to avidin. The lineage of avidin/streptavidin is still unknown.

Avidin is a tetrameric protein with one disulfide bond per subunit. The tetramer does not dissociate into monomers even in the presence of 8 M urea. The disulfide bonds are inaccessible to most reducing agents (Green, 1975).

Avidin is a glycoprotein with a heterogeneous carbohydrate chain (Bruch and White, 1982). Due to its high pl and the presence of sugar residues, it presents a high nonspecific adsorption to biotin, DNA, and other proteins. Several methods have been applied to overcome the nonspecific binding of avidin. These include the use of high ionic strength buffer (0.5 M NaCl), blockers (bovine serum albumin) or 200 MM α-methyl-D-mannoside (Duhamel and Whitehead, 1990). Alternatively, a modified avidin derivative, neutravidin, can be used to overcome the above problems.

Background of Neutravidin

Neutravidin is a deglycosylated avidin with two key features which dramatically reduce the nonspecific binding. First, the carbohydrate is removed under mild conditions during the purification process. Second, the pl of neutravidin is close to neutral pH. The specific activity and the association constant for biotin binding are identical between neutravidin and avidin (Hiller et al., 1957). The avidin used in most of the experiments in this dissertation is neutravidin due to the decreased non-specific binding it exhibits. Table 4 compares the properties of avidin and neutravidin.

Background of Biotin-avidin Interaction

The biotin-avidin interaction is one of the strongest known non-covalent interactions between protein and ligand. The binding dissociation constant (i.e., K_{D}=/K_{eq}) is about 10⁻¹⁵ M, which corresponds to a free energy of association of about 21 kcal/mol, a value in the same order of many covalent bond formations. For comparison, the K_{D} of topo II-DNA non-covalent binding is about 10⁻⁹ M, a million fold lower than the biotin-avidin interaction.

The biotin-avidin complex can withstand brief exposures of high temperatures up to 132° C. (Donovan and Ross; 1973), guanidine HCl up to 8 M, and SDS or Triton X-100 up to 1%. The biotin-avidin complex is stable from pH 2 to pH 13 (Green, 1975). Once avidin binds to biotin, the complex is essentially irreversible.

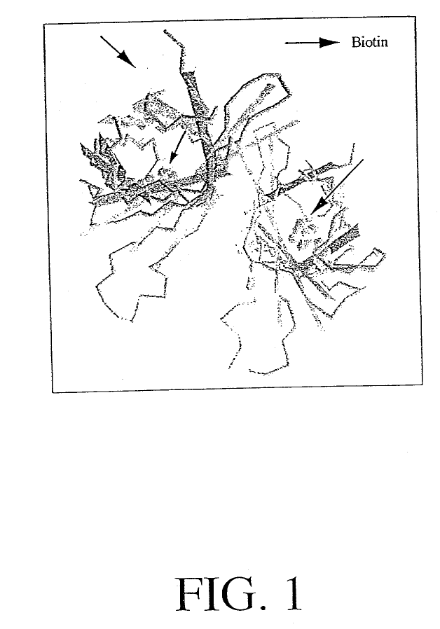

Avidin exists as a tetramer, and each of the four avidin monomer binds to one biotin. The crystal structure of Avidin reveals that it has a 2-fold symmetry with biotin binding sites in two pairs on the opposed faces of the molecule. FIG. 1 is the structure obtained from the Brookhaven Protein DataBank (PDB access code: 1 AVD).

| | 10 | 20 | 30 | 40 | 50 |
|---|---|---|---|---|---|
| 1 | MVHATSPLLL | LLLLSLALVA | PGLSARKCSL | TGKWTNDLGS | NMTIGAVNSR |
| 51 | GEFTGTYITA | VTATSNEIKE | SPLHGTENTI | NKRTQPTFGF | TVNWKFSEST |
| 101 | TVFTGQCFID | RNGKEVLKTM | WLLRSSVNDI | GDDWKATRVG | INIFTRLRTQ |
| 151 | KE | | | | |

Table 3 Sequence of avidin monomer with a signal peptide Signal peptide is from # 1 to #24. A disulfide bond forms between #28 (Cys) and #107 (Cys) residues. Asn (#41) is linked to a carbohydrate chain There is a variant residue at #58 (50% lie and 50% Thr). Biotin binding occurs at residue #57 (Tyr). The sequence is from GeneBank (accession number: P02701).

TABLE 4
Comparison between avidin and neutravidin

| | MW | pl | Carbohydrate | Non-specific binding |
|---|---|---|---|---|
| Avidin | 67 k.Da | 10.0 | Yes | High |
| Neutravidin | 60 kDa | 6.3 | No | Very low |

### Example 2

### Formation and Purification of Biotin-Avidin Networked Gene System (BANG)

Introduction

BANG formation via photobiotinylation

As explained in Example 1, the biotin-avidin interaction is one of the strongest known non-covalent interactions between protein and ligand. Furthermore, each avidin binds to four biotin molecules. Therefore, avidin can be utilized to crosslink DNA, as long as each DNA molecule has at least two biotins.

The first attempt at labeling DNA with biotin used the photobiotinylation method. Photobiotinylation, in which a derivative form of biotin is activated under strong visible light (350 nm range), is one of the simplest and least expensive ways of labeling DNA with biotin. Biotinylation occurs within 15 minutes, and the resultant biotinylated DNA can be purified by conventional ethanol precipitation.

BANG formation via PCR

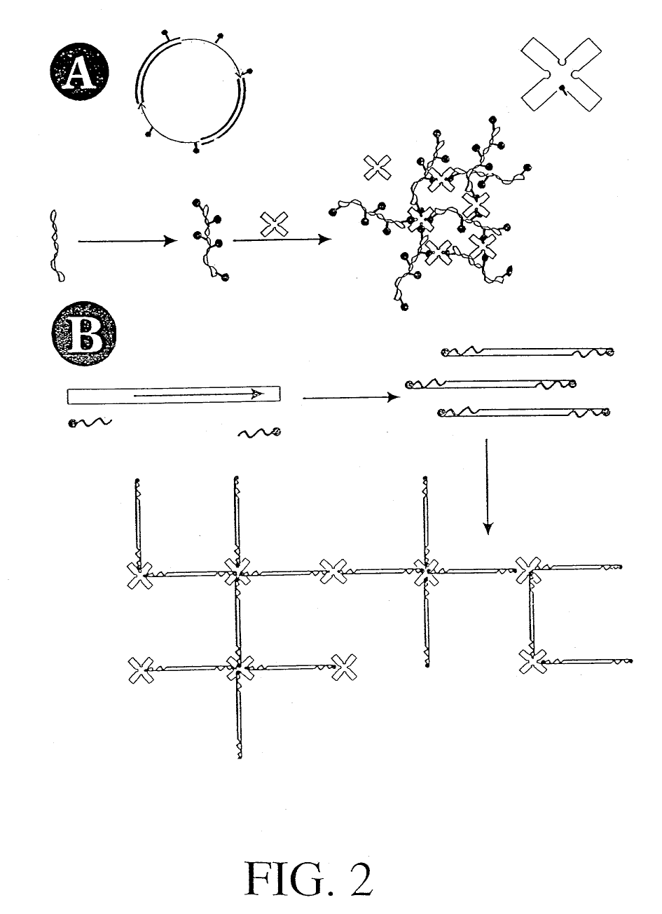

Photobiotinylation randomly labels DNA with biotin molecules. The site of biotinylation and the ratio of biotin vs. DNA molecules, However, were difficult to control. As a result, it formed huge DNA-DNA crosslinked networks that were even inaccessible to restriction enzymes and incapable of expression (see later in this EXAMPLE). This was probably due to the high degree of compaction and biotinylation at promoter and coding sequences (FIG. 2, panel A).

In order to obtain controlled biotinylated DNA molecules, a novel approach was devised (FIG. 2 ,panel B). Biotin end-labeled (5′) primers were used to amplify specific genes. After purification of PCR products, every single DNA molecule was attached with two biotin molecules at either end. DNA-DNA crosslinking was achieved by adding avidin molecules. Since each avidin bound to four biotins and each DNA molecule had two biotins at either end, a DNA-DNA network was formed via the aforementioned strong biotin-avidin interactions (FIG. 2 ).

To increase the efficiency of BANG formation, a multiple step seeding procedure was also devised. In this approach, a very limited amount of avidin was added first to create “seeds” for BANG DNA growth. Avidin was then added subsequently in steps, and the efficiency of BANG formation increased substantially. This multiple step seeding procedure is quite universal. It could be easily adapted to accommodate computer controlled automatic processes in future development of large scale BANG production . It could also be applied along with the GeneGrid Recycling of Avidin-Saturated Products (GRASP) purification scheme to produce GeneGrid (next section). Cycle purification (GRASP procedure)

As will be seen later in this chapter, BANG by PCR provided a novel way to crosslink DNA together via biotin-avidin non-covalent bonds. The degree of crosslinking, however, is heterogeneous. While in many applications this heterogeneous crosslinking does not cause any problems, occasionally a homogenous population of DNA-DNA networks is desired.

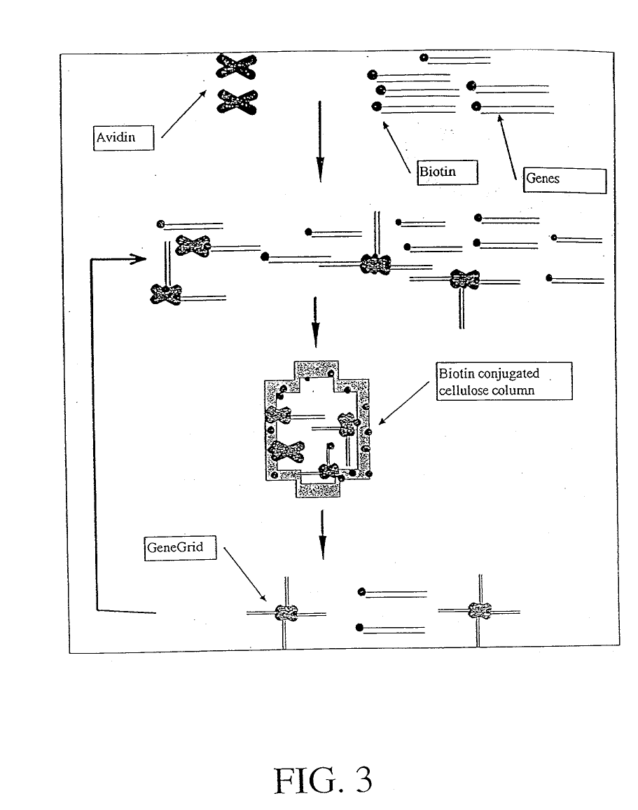

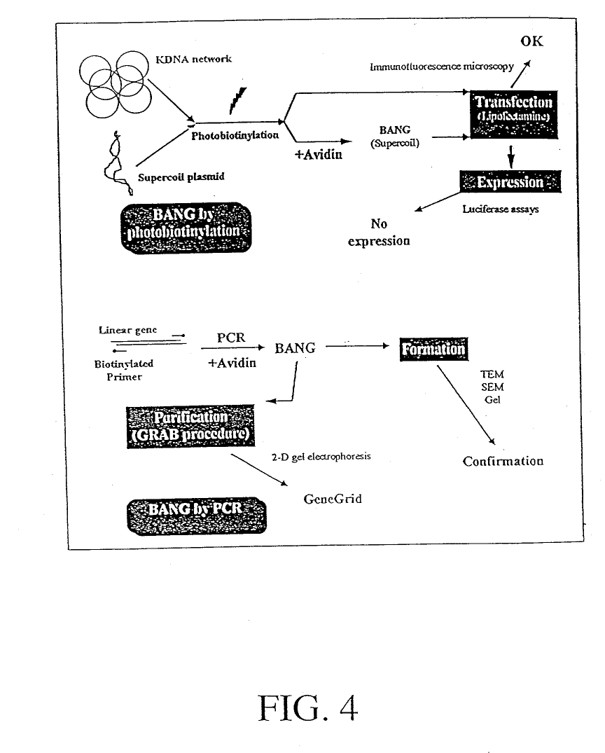

A novel recycling enrichment method was developed using a biotin conjugated cellulose column (FIG. 3). In this method only one primer of the PCR reaction was labeled with biotin at its 5 prime end. After PCR, the one-end-biotin-labeled DNA molecules were networked with a limited amount of avidin, which were effectively saturated. The reactions were then passed through a biotin-cellulose column where unsaturated avidin molecules were retained by biotin in the column. The flow-through contained two populations: one was made up of homogeneous avidin molecules saturated with four DNA molecules, whose free ends had no biotin (non-“sticky” ended); the other consisted of free, unbound DNA molecules, whose one free end had one biotin (“sticky” ended). The whole process was then recycled several times to enrich the first population. The final product was a homogeneous population which was termed the “GeneGrid”. The novel recycling enrichment procedure was called GeneGrid Recycling of Avidin-Saturated Products method (GRASP). The GeneGrid purified from the GRASP procedure could be used as a building block for the BANG system.

Specific Aims and Strategies

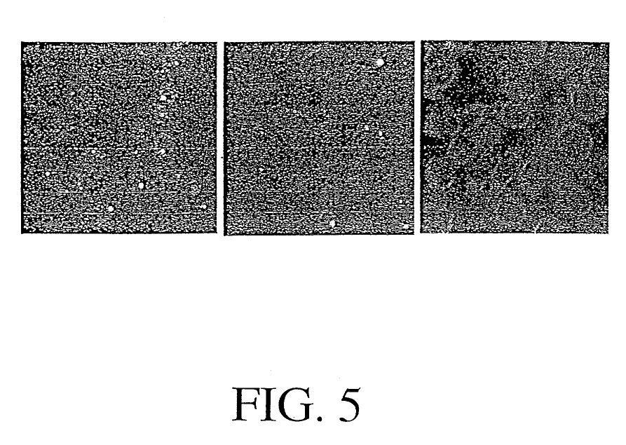

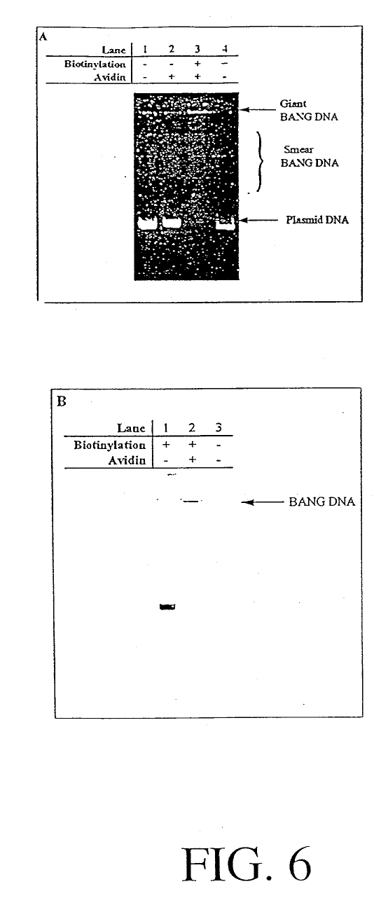

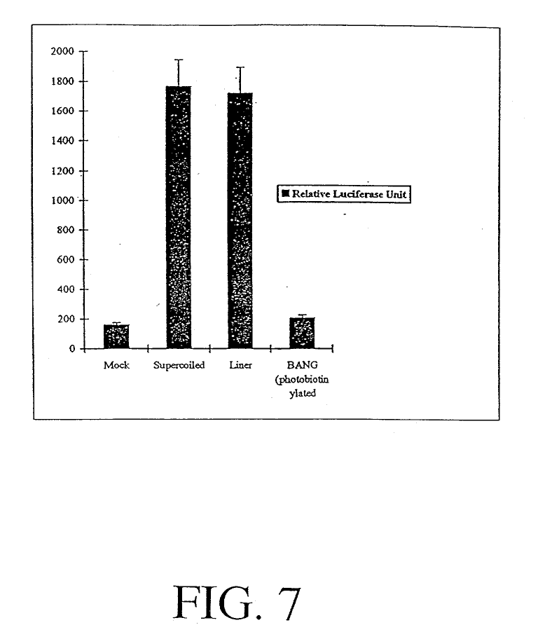

There are four specific aims of this chapter. The first two answer the following questions: One, can a huge DNA network such as kDNA be introduced into cells? And two, can supercoiled DNA crosslinked by photobiotinylation and avidin be expressed in vivo? Since the answer to the first question was yes (FIG. 5) and the answer to the second question was no (FIG. 6), the next two specific aims were to investigate a novel way to crosslink PCR generated linear DNA via biotin-avidin interaction and to purify the crosslinked BANG complexes.

The experimental strategies are illustrated in FIG. 4

Materials and Methods

Plasmids and primers

Plasmids used in this study include luciferase expression vector pGL2 and pGL3 series from Promega Inc. (Madison, Wis.), Green Fluorescence Protein (GFP) expression vector pGFP-c2 from Clonetech, (Palo Alto, Calif.), β-Galactosidase expression vector from Promega, and pTS-Luc from Dr. Lee Johnson's lab (Molecular Genetics Department, Ohio State University, Columbus, Ohio). Primers were synthesized and 5′ labeled with biotin by Life Technologies, (Gaithersburg, Md.).

Preparation of kDNA

Crithidia was cultured in BHI media (3.7% brain heart infusion, 20 μg/ml hemin in 0.5 mM NaOH) for 3 days until log phase before harvest by centrifugation. The culture was lysed by 2% Sakosyl with 1 mg/ml proteinase K at 37° C. Twenty minutes of centrifugation at 20,000 rpm separated the kDNA network in a CsCl step gradient solution.

Photobiotinylation

Equal volumes of DNA (1 μg/μl) and PHOTOPROBE® biotin were mixed and irradiated in an ice bath 10 cm below a mercury vapor lamp (wavelength around 350 nm) for 15 minutes. After photoactivation, an equal volume of 0.1 M Tris-HCl, pH 9.5 was added to deprotonate the non-reactive PHOTOPROBE® biotin. Then; the non-reactive PHOTOPROBE® biotin was extracted with 2-butanol. The final biotinylated DNA was purified by conventional ethanol precipitation.

PCR

PCR reactions were performed using the Advantage™ KlenTaq polymerise (Clontech Laboratories, Inc., Palo Alto, Calif.) according to the manufacturer's recommendations. The KlenTaq polymerise consists of a mixture of two polymerises to provide 3′→5′ proofreading activity: a 5′-exo-minus, N-terminal deletion of Taq DNA polymerise, and a Deep, Vent_{R}™ polymerise. It also contains neutralizing monoclonal antibodies directed against the polymerases for “hot start” PCR. Briefly. 1 ng of template DNA was amplified with 5 μm of each primer in a solution containing 2.5 mm of each deoxyribonucleotide and 1×KlenTaq reaction buffer (0.8 mM Tris-HCl, pH 7.5, 1.0 mm KCl, 0.5 mm (NH₄)₂SO₄, 0.1 mm EDTA, 0.5 mM, β-mercaptoethanol, 0.005% Thesit 1% and 1% Glycerol). Two-step thermal cycling was performed as follows:

| | | |
|---|---|---|
| 94° C. | 1 min. | 1 cycle |
| 94° C. | 30 sec. | 25 cycles |
| 68° C. | 3 min. | 25 cycles |
| 68° C. | 5 min. | 1 cycle |

The exact annealing/polymerizing temperature depended on the primer sequences. PCR products were optimized until one correctly sized band appeared on the agarose gel. All PCR reactions were purified using Qiaquick columns (Qiagen Inc., Santa Clarita, Calif.) before any further applications.

BANG formation

BANG formation was typically performed at room temperature unless specified.

Almost all the formation reactions were incubated in 1×BANG binding buffer (BBB: 5 mm Tris, 0.5 mm EDTA, 50 mm NaCl). Generally, a limited amount of avidin (1/100 of biotin) was added first in order to form “seeds” for BANG formation. More avidin was added sequentially at 30 minuteintervals. Generally, BANG formation was heterogeneous in terms of forming patterns except in the case of GeneGrid where the GRASP method was applied to enrich and purify the avidin-saturated products (see cycle purification.

Transmission EM

BANG DNA (10 ng at 1 ng/μl ) was incubated with 40 μl of 0.25 MNH₄Ac for 1 minute at room temperature before adding 1 μl of 1 mg/ml Cytochrome C (Sigma). The solution was then loaded onto a Formvar-coated grid. The grid was stained in uranyl acetate stain stock (one drop of uranyl acetate in 50% of EtOH) for 5 seconds. The grid was then air dried for at least. 15 minutes at room temperature. Finally, the grid was shadowed with platinum-palladium at a 10° shadowing angle on a rotary stage with a speed of 100 rpm. A Philips CML-5 electron microscope was used to view the DNA sample.

Scanning EM

BANG DNA (1 ng/μl) purified from an agarose gel was spread onto a clean metal specimen stub and dried in a vacuum. In some cases, freshly peeled mica was used to support the DNA before mounting it on a specimen stub using double-stick tape. The specimen was low-angle coated with platinum-palladium before being examined with a JEO-1 scanning electron microscope.

GRASP procedure

GRASP is a recycling purification method for the enrichment of the avidin-saturated biotinylated-genes (“tetramer-gene”, also termed as “GeneGrid”). Biotin conjugated cellulose resins (Pierce, Rockford, Ill.) were used to construct biotin columns, which retained any unsaturated avidin molecules. Two ml of the resins were typically used in a 2×5 mini-column, and chromatography was performed at room temperature by gravity. Fractions were then collected and assayed by electrophoresis.

2-D gel electrophoresis

Two dimensional electrophoresis was performed to identify the GeneGrid. The first dimension was run a neutral solution, and the second dimension was run under denaturing condition. SYBR-II dye was then used to detect the denatured single strand DNA using an epifluorescence UV gel documentation apparatus equipped with an orange-340 filter. Double strand DNA gave only background signals.

6.3 Results

Transfection of kDNA to Hela Cells by Lipofectamine

In the process of trying to mimic kDNA and to evaluate the applications of DNA-DNA crosslinking, an important question was whether or not a large DNA network such as kDNA could be introduced into mammalian cells. To answer this question, kDNA was first biotin-tagged by photo biotinylation. Various transfection methods including CaP0₄, electroporation, and lipofectamine were applied to introduce kDNA into the cells. The results were visualized and evaluated by avidin-conjugated fluorescine (FTTC) using indirect immunoflourescence (IF) procedures. While CaPO₄ and electroporation gave rise to poor transfection efficiency, lipofectamine seemed to successfully introduce kDNA into Hela cells with reasonable efficiency (FIG. 5 ). Hela cells transfected with photobiotinylized kDNA (left two panels) or without (right panel, mock) were fixed and permeablized as described in the Materials and Methods. Avidin conjugated FITC was used to visualize the photobiotinylized kDNA. In the mock transfected cells (right panel), only background fluorescence was observed. In the kDNA-transfected cells (left two panels), most labeling appeared around the peripheral region of the nucleus. The pictures also showed that the positive signals (i.e. FITC labeling) varied in size (comparing the upright part of the middle picture with the rest of the labeling). This was probably due to the different degree of biotinylation as photobiotinylation randomly labeled kDNA.

FIG. 30 shows Hela cells stained with avidin-conjugated FITC after biotinylated-kDNA transfection. Obviously, kDNA was presented in transfected cells (FIG. 5, left and middle panels), and was not seen in mock-transfected cells (FIG. 5, right panel). Therefore, biotinylated, giant DNA networks can be introduced into mammalian cells by lipofectamine.

Formation of BANG using photobiotinylation

Photobiotinyiation is a simple and inexpensive way of labeling nucleic acids with biotin. Photobiotinylation was performed using PHOTOPROBEO®, a photo-activatable form of biotin (Vector Laboratories, Burlingame, Calif.). It is an aryl azide derivative of biotin with a positively charged spacer arm between the biotin and the azide group. After mixing plasmid DNA (supercoiled form) with PHOTOPROBE® biotin and exposing it to strong visible light in the 350 nm-370 nm range (produced by a mercury vapor lamp) covalent binding of biotin to DNA resulted (Forster et al., 1985). BANG was then formed by incubating photo biotinylated plasmid DNA with avidin molecules as described in Materials and Methods. Electrophoresis was used to confirm the formation of BANG (FIG. 6, panel A). A DNA band with a huge size appeared (lane 3, immediately below the gel well), indicating a large DNA network was formed. To confirm that the band was indeed biotinylated, a southern blot of the gel was probed by avidin conjugated with alkaline phosphatase (FIG. 6, panel B). The results clearly confirmed that the band was biotinylated.

Expression of BANG Generated by Photobiotinylation

In the process of trying to crosslink DNA-DNA together, the second question asked was whether or not a large DNA network such as BANG generated by photobiotinylation could be expressed in vivo. Hela cells (2×105) were transfected by lipofectamine with BANG DNA (500 ng) generated by photobiotinylation. Cells were lysed 48 hours after transfection, and 20 μl of lysates were used for luciferase assays. The same amount (mole) of supercoiled plasmid DNA and biotinylated linear fragment were also transfected as positive controls (FIG. 7). The data suggest clearly that BANG DNA generated by photobiotinylation exhibits background expression of luciferase activities. This negative result cannot be due to experimental failure, since the positive controls worked; nor can it be interpreted as failure of transfection, since the same huge size kDNA was able to enter the cells by the same transfection procedure.

The most likely cause of this negative result is the high degree of compaction of DNA. As illustrated in FIG. 2 (panel A), biotin molecules were randomly labeled onto DNA molecules by photobiotinylation. This resulted in a huge network connected by avidin. Any attachment of avidin molecules at the core sequences (such as promoter and coding regions) would probably block transcription. Therefore, BANG generated by photobiotinylation is not suitable for gene expression.

Formation of BANG using PCR

To avoid the photobiotinylation problems of random and uncontrollable attachment of biotin to DNA molecules, a simple and straightforward approach was used. Primers whose 5′ ends were conjugated to biotin were applied in a PCR reaction to produce a large quantity of biotinylated DNA. Avidin was then added to form BANG DNA.

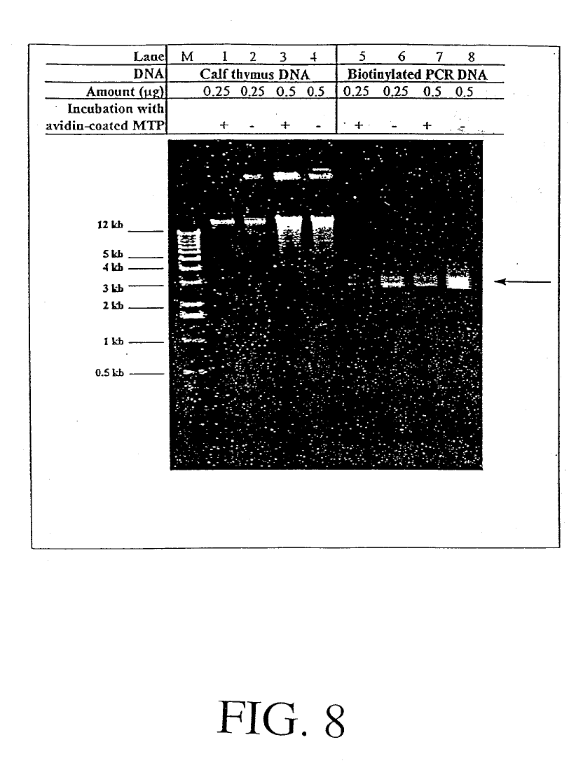

Since the biotinylated PCR products were essential in the formation of BANG DNA, avidin-coated multiple-well titration plates (MTP) were utilized to check the efficiency of biotinylation of the PCR products. If most of the products were biotinylated, incubation in the avidin-coated MTP would result in depletion of PCR products (i.e., monomer). FIG. 8 shows the expected depletion of biotinylated PCR monomers (FIG. 8, lanes 5 and 7). Calf thymus DNA was used as a non-biotinylated DNA control (negative controls). No depletion was observed (FIG. 8, lanes I and 3).

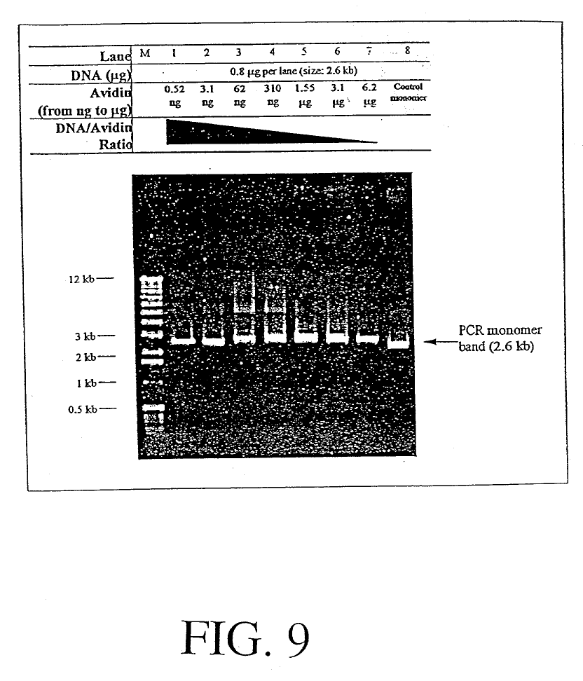

After the confirmation that the PCR products were indeed biotinylated, BANG formation was performed ( FIG. 9). Briefly, 800 ng of purified PCR products (monomer DNA with both ends labeled with biotin) were incubated with various amounts of avidin at room temperature for 2 hours. The reactions were then loaded onto a 0.8% agarose gel and electrophoresis was carried out at 50 volts for 45 min. The gel was finally stained with EtBr for documentation. Lane M was the 1 kb ladder shoving the DNA molecular weight references. Lane 8 was the control where no avidin was added (no BANG formed). From lane I to lane 7 avidin was added in increasing amounts. It was clear that higher molecular weight bands formed at least in lanes 2, 3, 4, 5, and 6, suggesting that BANG DNA was created. In lanes 1 and 7. where the relative number of avidin molecules was either too many or too few compared to DNA molecules, no higher molecular weight bands were observed. This could be explained as follows. It's known that biotin-avidin interaction is essentially irreversible. Hence, once avidin binds to biotin, it stays bound. Therefore, at the ratio where avidin molecules were too few (lane 1 situation), most of the avidin molecules were saturated at once by biotinylated DNA. Consequently, there were too few avidin available for BANG formation. On the other hand, at the ratio where avidin molecules were too many (lane 7), the majority of the biotinylated DNA molecules were bound immediately by avidin. Therefore, there were too few free biotinylated DNA left for the BANG structure.

FIG. 9 reveals the expected BANG formation pattern at various avidin concentrations. At either extreme of the ratio of DNA was avidin (lane 1 or 7), no or little BANG formation was detected on the agarose gel. Nevertheless, at certain ratios of DNA vs. avidin (lane 3, for example), an estimated 50% of the input monomers were shifted to the smear BANG DNA. Many attempts were made to increase the weld of BANG DNA, such as a greater range of titration of avidin molecules, longer time incubation, different buffer solutions, etc. None of them seemed to be able to exceed the 50% efficiency of BANG formation (data not shown). An alternative approach, however, was created which increased the efficiency to at least 80% (see next section). Formation of BANG using PCR and a seeding procedure

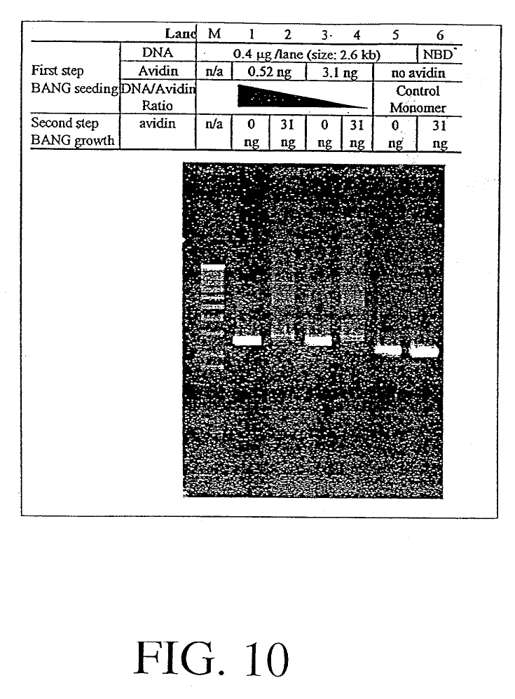

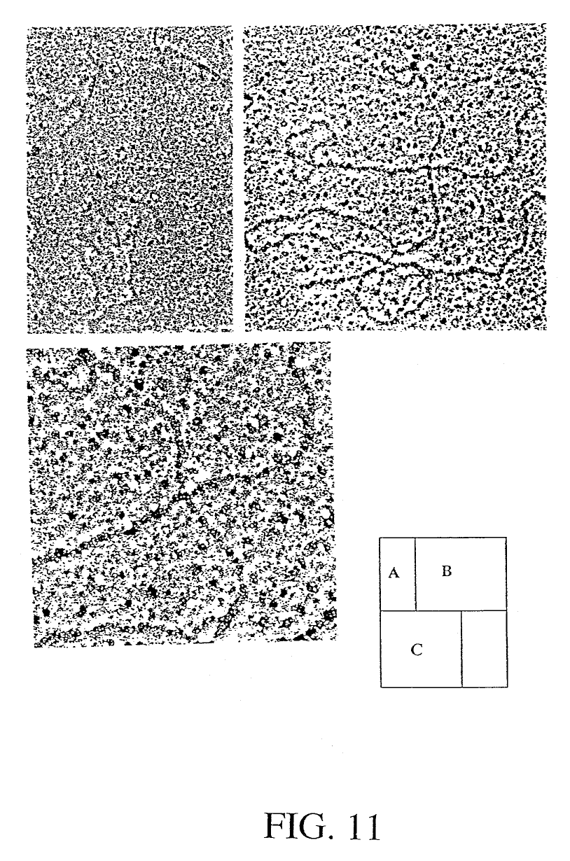

As seen from FIG. 9 (above), BANG formation was largely dependent on how many avidin molecules were added. FIG. 10; on the other hand, illustrates that BANG formation was also dependent on how often avidin molecules were incubated with, the biotinylated genes.

To increase the efficiency of BANG formation, avidin was first incubated with biotinylated DNA in a very limited amount (1 avidin molecule per hundreds of DNA molecules). Most of the avidin molecules would be saturated by the biotinylated DNA so that “seeds” would be created. At this point, as expected, no BANG was detected. Then, more avidin was applied in which the seeds in the first step would “grow” along with other newly formed avidin-biotin networks. This two-step procedure greatly increased the efficiency of BANG growth. In FIG. 10, the seeding steps are shown in lanes 1 and 3 (no BANG DNA were observed). However, the second step addition of avidin (lanes 2 and 4) shifted most of the monomers to a higher molecular weight, indicating large amounts of the DNA were incorporated into the BANG structure. Negative controls were in lanes 5 and 6. In lane 5, no avidin was added, and in lane 6 was non-biotinylated DNA (NBD). Neither lane shows any smear in the higher molecular weight area on the gel.

Visualization of BANG formation by TEM

Even though the above electrophoresis experiments provided strong evidence suggesting that biotin-avidin networked DNA had been created, the ultimate evidence should be at the molecular level where actual crosslinkings (“crossed” or “branched” DNA molecules) can be observed. Electron microscopy (EM) was performed to obtain such evidence.

FIG. 11 shows the transmission electron microscopy (TEM) pictures of BANG DNA. Biotin-labeled DNA molecules (size: 2.58 kb) were obtained by PCR and prepared for EM as described in Materials and Methods. Panel A is the control monomer DNA where no avidin was added. The average contour length of these monomers was about 43 mm. Considering the EM magnification factor (45,000×) and the print reducing scale (1 print cm=0.87 cm), this length corresponded to 0.83 μm which was very close to the expected size (2.58 kb DNA was about 0.88 μm). Panels B and C are BANG DNA under the TEM at different magnification. Clearly, the crosslinked structure are observed. The length of each strand was measured and calculated as above. The results were very close to the expected size (within ±10% error). Therefore, these “branched” crosslinked DNA molecules provided conclusive evidence that the BANG structure had been formed.

Visualization of BANG Formation by SEM

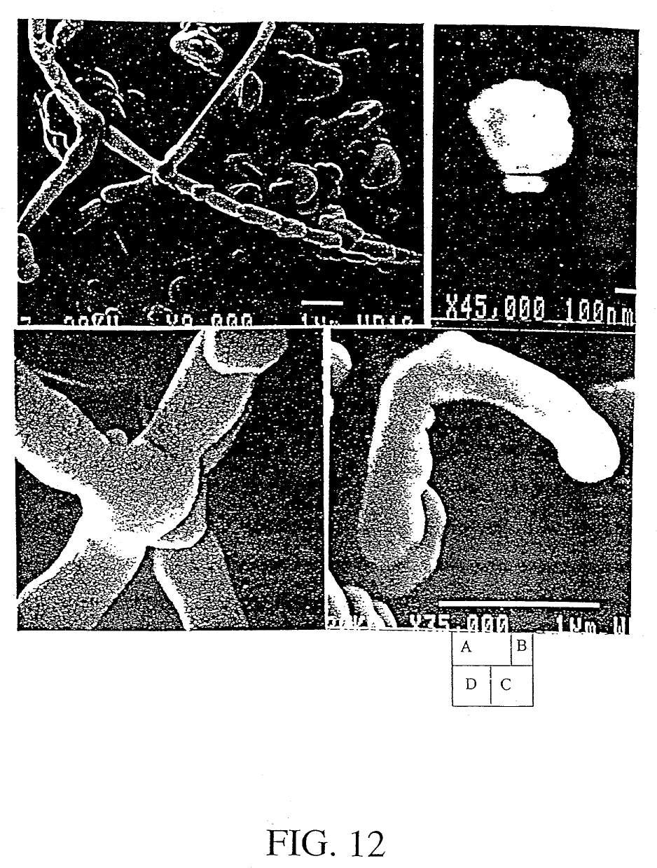

To further confirm the above TEM results, scanning electron microscopy (SEM) was also applied to visualize the BANG structure. DNA was prepared and coated as described in the Materials and Methods. FIG. 12 shows BANG DNA observed by SEM: Under the coating condition used in this study, the width of the DNA (diameter of the helix) was widened enormously (Amrein, et al., 1988; Amrein, et al., 1989). Nevertheless, the alteration due to the coating was minimum for the contour length of DNA.

Panel A shows a BANG structure with polymers in each crossed arm. Notice the repeated fragment (probably due to non-complete coating) which is about 0.8 μm long, corresponding to 2.58 kb DNA molecules. Panel D is a blow-up version of panel A, showing the details of the crosslink site. Clearly one can see a bulge at the crosslink site which is presumably an avidin molecule. Since the size of such a bulge is about 600 nm, much bigger (133 fold) than the expected avidin molecule (around 4.5 am, Green, et al., 1971), it could be argued that the whole structure was neither DNA nor avidin but artifacts. To prove that the bulge showing here was indeed an avidin molecule, a pure avidin solution was dried on the stud and coated exactly as in panel D. Avidin molecules were then viewed at the exact same magnification (45000×). Panel B is the SEM picture of pure avidin molecules which reveals them to be almost exactly the same size as the bulge shown in panel D. Notice that in this case one is even able to discern four subunits strongly suggesting that the images in panel A and D are indeed avidin molecules. The enlarged incorrect size could be explained by artifacts from the coating procedure, since the DNA width was also enlarged to a similar order (about 160 fold increase). In summary, both TEM and SEM pictures proved that the branched, networked BANG DNA were indeed created.

Effect of Temperature on BANG Formation

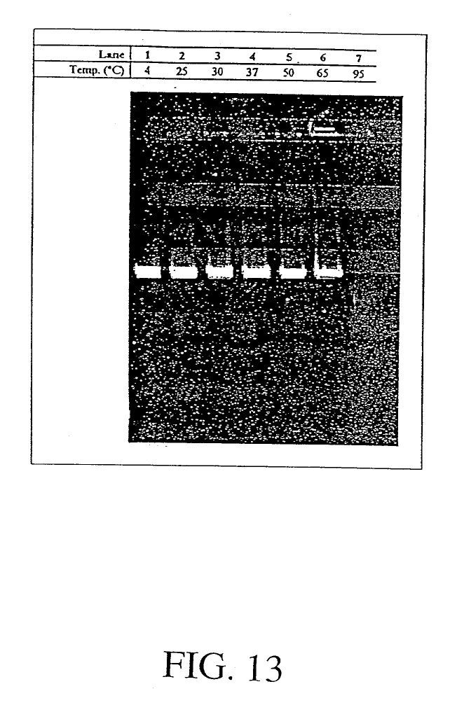

To further investigate BANG formation, a temperature titration of the reactions was performed. Biotinylated DNA was incubated with avidin (without the seeding procedure) at different temperatures for 30 min. The reactions were immediately loaded onto an agarose gel. FIG. 13 shows the temperature titration results. Few BANG formations were observed when the reaction was incubated at 4° C. No BANG formation was evidenced when the formation was carried out at 95° C. (DNA degradation occurred). BANG formation was observed from 25° C. to 50° C. with a very slight increase of efficiency towards higher temperatures. At 65° C., however, a major, huge band appeared close to the well along with the usual BANG DNA smear. The nature of this giant size band is uncertain. One plausible explanation is that incubation at 65° C. for 30 min. produced single strand DNA (ssDNA) which tend to be sticky to each other due to DNA complementation. Large amounts of ssDNA could have easily increased the size of BANG DNA. In a way, this is similar to the photo-BANG scenario where DNA is bodily labeled with “sticky” biotin (FIG. 3).

GeneGrid purification by GRASP procedure

BANG DNA were crosslinked by biotin-avidin interactions. The degree of crosslinking, however, was heterogeneous and difficult to control. In other words, each avidin molecule in a BANG system could be in one of five states: totally unsaturated (bound to 0 biotin), totally saturated (bound to 4 biotin molecules) or partially saturated (bound to 1 or 2 or 3 biotin molecules). A novel recycling enrichment method (GRASP) was developed using a biotin conjugated cellulose column (FIG. 2 ) to purify BANG DNA with totally saturated avidin (GeneGrid). In this process, one-end biotinylated PCR products were used to generate BANG DNA by the seeding procedure. After several rounds of enrichment by GRASP, the final products were examined in a two dimensional agarose gel. The first dimension was run in a neutral solution, and the second dimension was run at the denaturing condition. SYBR-II dye, which only stained single stranded nucleic acids, was then used to detect the denatured single strand DNA.

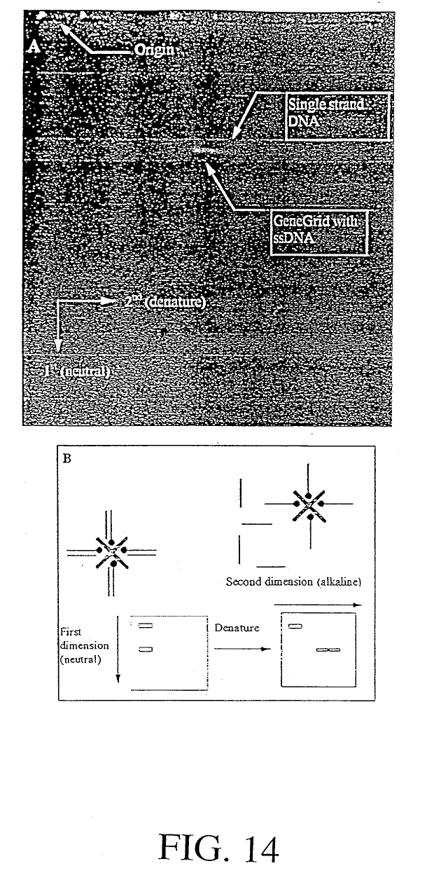

The double strand DNA were denatured on a two-dimension gel with the second dimension running under the denaturing condition. Because only one end of the DNA was biotinylated and hence physically attached to the avidin molecule, the denaturing condition resulted in two species: single strand DNA and GeneGrid with ssDNA (FIG. 39B). After several rounds of passing through the biotin-column, the majority population was the saturated avidin (i.e., with four biotinylated genes attached). FIG. 14 A shows the expected two major bands on a 2-D gel, indicating that the GRASP procedure indeed enriched BANG DNA with saturated avidin (GeneGrid).

Discussion

Formation of BANG DNA

Since each avidin molecule irreversibly binds to four biotin molecules, crosslinked DNA-DNA could be created through biotin-avidin interaction, provided that DNA molecules are biotinylated. Biotinylation of DNA molecules could be achieved either by photobiotinylation (photo-BANG) or PCR (PCR-BANG). Photo-BANG, however, generated huge, highly compacted, BANG structures which were unable to express genes. BANG formed by PCR, on the other hand, were designed to utilize the amplification power of PCR and non-covalent bonds of biotin-avidin to bring the same or different DNA constellations together. The crosslinked structures were confirmed by electrophoresis, TEM, and SEM. BANG DNA were also proved to be accessible, stable, and expressible (see below).

The two step seeding procedure was designed to increase the efficiency of BANG formation. In addition, it could also be adapted to multiple steps which could provide a way for future computer-controlled large scale production of BANG DNA. For example, the first seeding step could be split into 100 steps in which each step contains one hundredth of the amount of the avidin molecules used in the two step procedure. This should increase the seeding efficiency greatly, thus increasing BANG formation dramatically.

The topological states of BANG DNA is another issue which needs to be addressed. The above reactions (FIG. 6)were also run on an E Br gel, which gave essentially identical results (data not shown), suggesting that there were no topological isoforms existing in the BANG system. Since only one strand of each DNA molecule was actually biotinylated (5 prime), only one strand was physically attached to the anchoring avidin molecule, leaving a free-end on the other strand. In other words, there was always one strand with a free end which could rotate to relieve any topological constraint.

Purification of BANG

GRASP is a novel procedure developed to purify GeneGrid. This procedure is simple, fast, and universal. The method itself does not require any additional equipment except for conventional columns. The whole procedure takes several hours. Furthermore, the whole GRASP process could be easily adapted to a fully automatic operation using computer-assisted chromatography such as FPLC.

The flow-through from GRASP consists of mostly pure GeneGrid (tetramer) and a few monomers. The remaining monomers could easily be removed by incubating them in an avidin-coated multiple well plate (MTP).

GeneGrids as Building Blocks of BANG

GeneGrids purified by GRASP could be used as building blocks to form the BANG system. For example, GeneGrid containing the neomycin resistant gene (Neo Grid) could be used in any further BANG DNA formations. Also, different GeneGrids could be simply ligated together to form BANG DNA consisting of multiple genes. Restriction enzymes and/or biotinylated adapters could also be added to generate sticky ends to facilitate further downstream BANG formations. Besides GeneGrids. PromoterGrids could be generated in advance and in bulk using only promoter sequences. A similar strategy could also be applied to enhancers or other non-coding sequences (such as replication origination sequences,) to form EnhancerGrids, etc. In summary. GeneGrids are universal, yet flexible.

Advantages of BANG

There are at least five advantages of the BANG system. First, DNA fragments are brought together by a non-covalent interaction. Therefore, same or different gene constellations can be readily formed without frame-shift problems. Second, combined with DNA ligases, these same or different genes can be arranged under different promoters and/or enhancers without conventional subcloning. Third, unlike most plasmids where at least several kilobases of bacterial sequences are needed in order to maintain their propagation, the DNA-DNA crosslinked network is formed totally in vitro so that only sequences of interest are necessary. Fourth, only the end(s) of a gene is labeled by biotin, therefore, the DNA-DNA network still possesses DNA characteristics, such as DNA denaturation, restriction digestion, ligation, etc., which makes the BANG system very versatile. Fifth, the principle of BANG can be readily applied to other macromolecules such as RNA and lipids, which will make BANG application universal.

A more detailed discussion of the advantages and potential applications of BANG, is presented in the last examples.

### Example 3

### In Vitro Characterization of Bang System

Introduction

Accessibility of BANG DNA

The BANG system provides a novel way to bring different genes together through non-covalent bonds. A critical question is whether or not these genes crosslinked by BANG are still functioning, since future applications of the BANG system will be largely determined by the accessibility of BANG DNA to various enzymes and/or enzyme complexes, such as transcription machinery.

Results in the previous example revealed that the BANG system formed by photobiotinylation (photo-BANG) could not be expressed in vivo. Further evaluations suggested that photo-BANG could not be digested by restriction enzymes either (data not shown). These results were not unexpected since the plasmids in the photo-BANG were probably highly compacted aced crosslinked (see above).

The previous example also described a novel, alternative way of forming BANG by using PCR (PCR-BANG). In this approach, linear DNA (rather than supercoiled plasmids) were used and the DNA strands were end labeled by biotin (rather than randomly body-labeled). Both TEM and SEM pictures revealed that BANG DNA were not compact at all. Therefore, it was expected that the accessibility of the BANG system to proteins and/or protein-complexes should be comparable to the monomer DNA, and that the level of gene expression (per mole) of BANG DNA in vitro should also be comparable to the monomer DNA.

Two aspects of BANG DNA were of special interests in the accessibility study. First was the accessibility of restriction enzymes to GeneGrid DNA. Restriction enzymes provided a defined system in which the outcome could be evaluated easily. They also could provide additional DNA manipulating tools for the BANG system in future applications. Second was the accessibility of transcription/translation machinery. The over-expression potential of the BANG system is one of its most exciting applications. In vitro transcription coupled with in vitro translation provided a necessary test for later in vivo expression study.

Stability of the BANG system

While the accessibility study was focused on the DNA component of the BANG system, the stability study was focused on both components of the BANG system: avidin and DNA. Two factors were of special interest in the stability study. First was the stability of the BANG system at different temperatures. Even though most of the in vivo applications would occur at 37° C., it would be very useful if the BANG system could tolerate higher temperatures, since higher temperatures would provide yet another way to manipulate the DNA components in the BANG system (such as denature/renature, hybridization, etc.). Second was the stability of the BANG system in the presence of proteinase K. This study was focused on the avidin part of the BANG system. Since the biotin-avidin interaction was extremely stable, the stability of the BANG system was expected to be very high. It was untested, however, as to whether or not the avidin could withstand the proteinase K treatment in vitro. SDS was not used in this study since the focus was not on trying to find a way to destroy the BANG system; rather, the focus was on assessing the stability of the BANG system in the presence of a proteinase.

Specific Aims and Strategies

The specific aims in this study were twofold. First was to evaluate the accessibility of the GeneGrid in vitro. This was achieved by examining the accessibility of restriction enzymes or the transcription/translation machinery. Second was to investigate the stability of the BANG system in vitro. This was achieved by testing the stability of the BANG structure at different temperatures and in the presence of proteinase K.

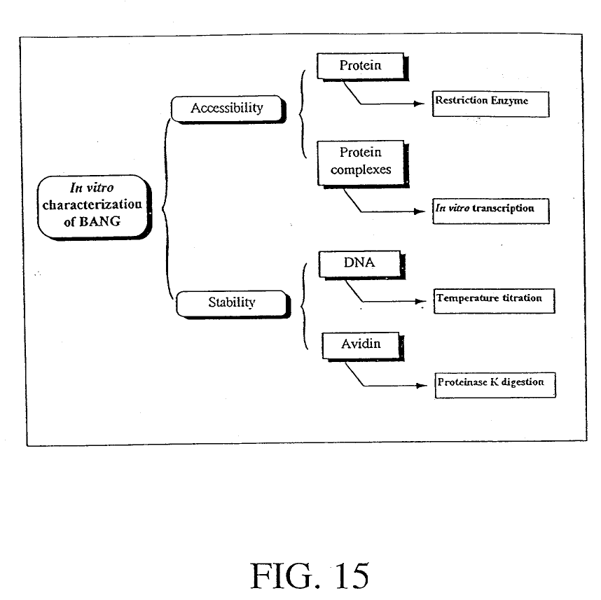

FIG. 15 shows the schematic illustration of the strategies of in vitro characterization of the BANG system.

Materials And Methods

Restriction enzyme digestion

GeneGrids were prepared without the GRASP purification procedure as described above. Restriction enzyme digestion were carried out at 37° C. for 2 hours according to conventional methods (Sambrook et al., 1989). The reactions were stopped by incubating them at 65° C. for 10 minutes before electrophoresis.

Temperature titration

An equal amount of BANG DNA was aliquoted into 7 tubes and then incubated at different temperature (95° C., 75° C., 65° C., 37° C., 30° C., 25° C., 4° C.) for 15 min. Electrophoresis was performed immediately after incubation.

Plasmids and Primers

Luciferase monomers (under the T7-promoter) were from PCR using a template of pGEM-Luc (Promega, Madison, Wis.) with two primers:

B-T7LUC-UP and B-T7LUC-DOWN, whose sequences were:

biotin-CCAATACGCAAACCGCCTCTCC, and

biotin-GAGCAGATTGTACTGAGAGTGCACC, respectively. Luciferase monomers (under the SV40 promoter) were from PCR using a template of pGL3-control (Promega, Madison, Wis.) with two primers: B-GL3UP-RV3

and B-GL3DOWN-RV4, whose sequences were:

biotin-CTAGCAAAATAGGCTGTCCC, and biotin-GACGATAGTCATGCCCCGCG, respectively.

In vitro transcription and translation

In vitro transcription/translation was carried out using the TNT® T7 coupled transcription/translation system from Promega Inc. (Madison, Wis.). The luciferase gene was cloned into a T7 promoter vector (pGEM). PCR reactions were performed using pGEM-Luc as a template to generate biotinylated products. BANG DNA was formed as above. PCR DNA (monomers) were used as controls. Reactions were incubated at 30° C. for 60 min. before luciferase assays.

Proteinase K Digestion

PCR-based BANG DNA were generated at different degrees as described before. They were then incubated with proteinase K (final concentration: 100 μg/ml) at 37° C. for 2 hours. Monomer DNA was used as a control. Agarose electrophoresis was performed to examine the effect of proteulase K digestion.

Results

Accessibility of Restriction Enzymes

To assess the accessibility of the restriction enzymes to the BANG system, GeneGrid DNA (instead of BANG DNA) was used as a model in the restriction digestion reactions. Due to both ends being “sticky” (i.e. biotinylated), the orientation of DNA molecules in the BANG system is heterogeneous. Thus, the restriction digestion results of BANG DNA were difficult to analyze. On the other hand, GeneGrid DNA provided a simplified model for the interpretation of restriction digestion results. GeneGrids were generated by PCR products with only one end labeled with biotin. Thus, they provided a necessary polarity in the DNA molecules which is essential for final digestion analysis.

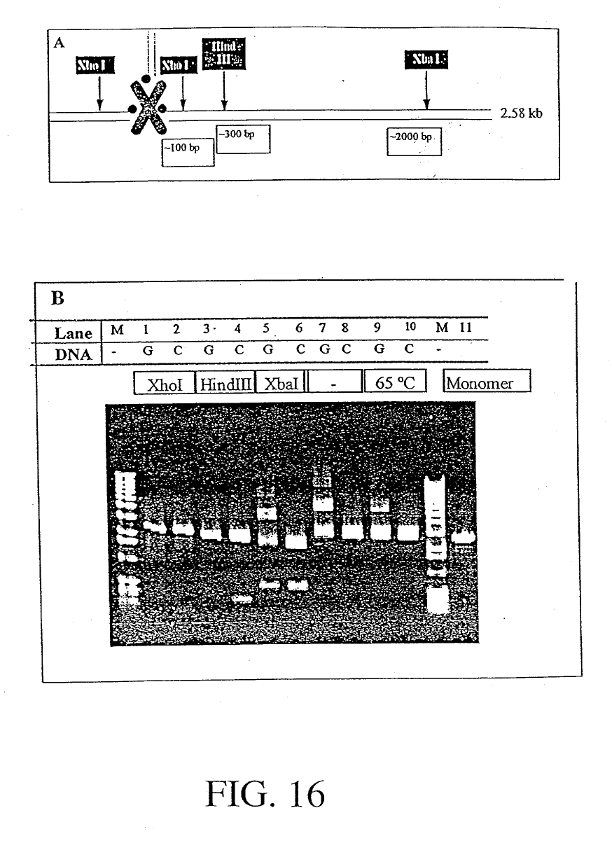

Three restriction enzymes were chosen: two were near the anchoring avidin (Xho I and Hind III) and one was near the free end (Xba I). FIG. 16A illustrates the expected results of restriction digestion. In the case of Xho I or Hind III, a major band of 2.4 kb or 2.2 kb, respectively, was expected to be present (FIG. 16B, lane I and 3, compare with the control DNA: lane 2 and 4). When Xba I was used, a small size band (500 bp) appeared (FIG. 16B, lane 5 and 6). In the meantime, the remaining GeneGrid sizes were reduced accordingly (compare lane 5 with lane 7). These results clearly indicated that restriction enzymes had full accessibility to the GeneGrid DNA.

In Vitro Transcription and Translation of BANG

To test whether or not the genes in the BANG system could be accessed by transcription/translation machinery, an in vitro transcription coupled with in vitro translation system was applied to assay the in vitro expression of a reporter gene (luciferase) in the BANG system. The luciferase gene under the T7 promoter was amplified by PCR with biotinylated primers. The same amount of DNA was then split into three tubes: two for the formation of BANG (with or without seeding procedure), and one for the control (monomer, no avidin was added). The in vitro transcription coupled with in vitro translation was carried out at 30° C. for 1 hour using TNT® T7 coupled with reticulocyte lysate (Promega, Madison, Wis.). A mock reaction (everything except the DNA templates) was included for negative control. The reactions were then diluted 1 to 10 and 1 to 1000. They were then assayed for luciferase activities. Notice that all the reactions contained the same amount of DNA. If the accessibility of the transcription/translation machinery to BANG DNA were hampered, then it would be expected that the final gene products (luciferase) would decrease dramatically. On the other hand, if the accessibility to BANG DNA were similar to the monomer DNA, then the luciferase activity should be comparable. Table 5 detailed the in vitro transcription/translation experiment which clearly demonstrated that the in vitro expression of luciferase by BANG was comparable to that of the monomer, strongly suggesting that at least in vitro, the BANG system did not reduce the accessibility of the coupled transcription/translation machinery to the genes.

| | | | | |
|---|---|---|---|---|
| Reactions | 1 | 2 | 3 | 4 |

First step BANG seeding

| 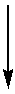 | T7-luciferase DNA | 0.99 μg | 0.99 μg | 0.99 μg | — |
|---|---|---|---|---|---|
| | Avidin | 0.09 ng | 0.09 ng | — | 0.09 ng |
| | | 37° C., 2 hours | | | |
| Second step BANG growth | | ↓ | | | |
| 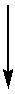 | Avidin | — | 54 ng | — | 54 ng |

| | 37° C., 2 hours | | |
|---|---|---|---|
| | ↓ | | |
| BANG formation | 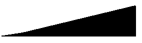 | Monomer Control | Negative Control |

In vitro transcription/translation

| | | | | | |
|---|---|---|---|---|---|
| 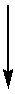 | TNT ® Lysates | 40 μl | 40 μl | 40 μl | 40 μl |
| | 1 mM Methionine | 1 μl | 1 μl | 1 μl | 1 μl |

| | 30° C., 1 hour | | | |
|---|---|---|---|---|
| Luciferase Assay | ↓ | | | |
| Luciferase RLU (1:10 of the reactions) | 32161724 | 28534134 | 33622996 | 132 |
| Luciferase RLU (1:1000 of the reactions) | 304613 | 369988 | 403727 | 216 |

Table 5 Accessibility of the transcription machinery to BANG DNA Experiment procedures were detailed in the left column of the table. Final luciferase remits were presented in the last two rows. Reactions 3 and 4 were controls (monomer control and negative control respectively).

Effect of Temperature on Stability of BANG

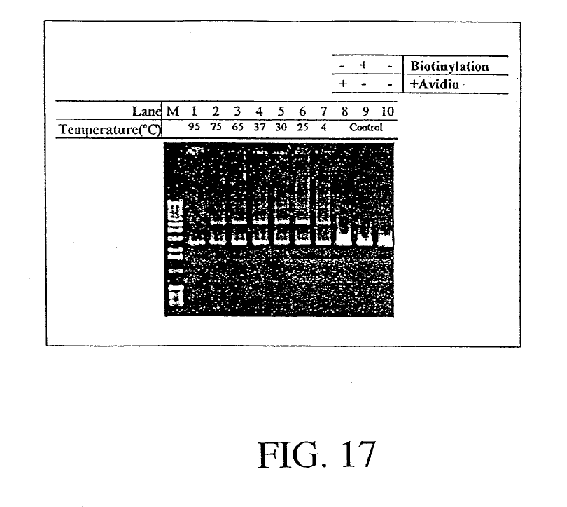

In the previous example, titration of temperatures was performed for the formation of the BANG structure. However, the effect of temperature on the stability of BANG after its formation was not examined. As can be seen in the above section, the BANG system was quite stable once formed. Taking into account that the biotin-avidin complexes could withstand brief exposures of high temperatures up to 132° C. (Donovan and Ross, 1973), it was expected that the BANG structure could tolerate as high a temperature as its DNA components. FIG. 17 shows temperature effect on the stability of BANG DNA. Lanes 8, 9, and 10 were controls at room temperature for BANG formation. Biotinylated DNA were generated by PCR and incubated with avidin as before to form BANG structure. Equal amount of DNA were then split into 7 tubes and incubated at different temperature for 15 min. before electrophoresis (lanes 1 to 7). The BANG structures were either abolished (lane 1) or decreased (lane 2) after a brief exposure to 95° C. and 75° C., respectively, presumably due to the destruction of the DNA molecules. BANG DNA were essentially unchanged from 4° C. to 65° C. (lanes 3, 4, 5, 6, and 7).

Effect of Proteinase K on the Stability of BANG

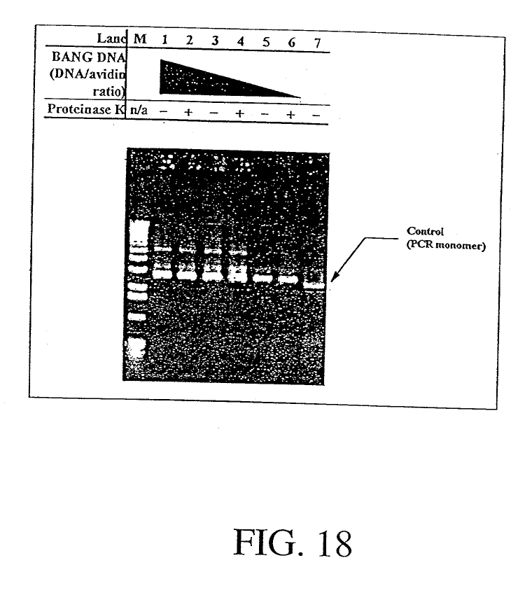

To investigate whether or not the avidin in the BANG system could be accessed by proteinase in vitro, a final concentration of 100 μg/ml of proteinase K was added after BANG was formed. The digestion reaction was incubated at 37° C. for 2 hours before electrophoresis. The PCR produced monomer DNA was used as a reference (FIG. 18, lane 7). Under the above conditions, proteinase K failed to digest avidin molecules (comparing FIG. 43 lanes 1, 3, and 5 to lanes 2. 4, and 6, respectively), suggesting; that either the avidin was resistant to the digestion of proteinase K or the avidin was protected by biotinylated DNA. (Proteinase K exhibits higher enzymatic activity in the presence of SDS and at 50° C. However, these conditions were not as relevant to the in vivo situations as 37° C. without detergent.) Considering the nature of biotin-avidin interactions and the results of the above experiments, it is conceivable that the crosslink sites in a BANG system are very stable both in vitro and in vivo, while the DNA components are still accessible to most of the proteins.

Discussion

Accessibility of BANG

The restriction enzyme digestion experiment strongly suggested that GeneGrid DNA was accessible to enzymes. Although GeneGrids are only building blocks of BANG DNA, the above results at least revealed that part of the BANG DNA could be accessed by enzymes. This was the first step toward final assessment of the accessibility of the BANG system. Further assessment of the accessibility came from the in vitro transcription coupled with in vitro translation. The BANG system does not seem to interfere with the functions of transcription/translation machinery in vitro suggesting that the genes in the BANG system might be able to express in vivo.

The accessibility of BANG is important, not only because it is necessary for BANG expression both in vitro and in vivo, but also it provides additional means to manipulate the DNA components in the BANG system. For example, combined with restriction enzymes and ligases, different GeneGrid could be ligated together.

Stability of BANG

Based on the nature of biotin-avidin interactions, it is not surprise to predict that the stability of the BANG system is very high. Two components, DNA and avidin, were examined for their tolerance toward high temperature and proteinase treatment, respectively. The results strongly support the notion that the DNA components in the BANG system maintain their nucleic acid characteristics while the avidin components are protected by proteinase at the physiological conditions.

The significance of BANG's high temperature (up to 65° C.) is that high temperatures provide yet another tool to manipulate the DNA components in the BANG system. For example, many enzymatic reactions could be employed at 37° C. and then stopped at 65° C. for 10 min. Hybridization could also be carried out without destroying the BANG system.

The fact that avidin molecules were protected from proteinases suggests an in vivo stability of the BANG system. This stability is necessary for achieving its over-expression potentials and non-covalent cloning. The following two example focus on the in vivo characterizations and the potentials of the BANG system.

### Example 4

### In Vivo Characterization of Bang Introduction

In vivo studies of the BANG system

As can be seen in previous examples, DNA-DNA crosslinking can be achieved by biotin-avidin interaction. The formation of the BANG system was confirmed by electrophoresis and by both SEM and TEM. The BANG system could be purified using the GRASP procedure to produce GeneGrid. In addition, in vitro studies revealed that the BANG system was quite stable. The DNA components in the BANG system were accessible to various restriction enzymes while the avidin component was protected from proteinase K digestion at the physiological conditions. Finally, the in vitro transcription/translation experiment suggested that the transcriptional machinery could express the genes in the BANG system.

The in vivo characteristics of the BANG system, on the other hand, were not evaluated. For example, could the BANG system be delivered into cells? If yes, what's the efficiency of delivery? Could the obvious gene dosage effect of the BANG system be utilized to achieve over-expression? Would the BANG system itself alter the growth of the cell? Could BANG DNA be integrated into the genome without loosing its gene dosage effect? To address these questions, in vivo characterization of BANG was carried out at two levels: the individual cell level and the population cell level.

Specific Aims and Strategies

To characterize the in vivo behavior of the BANG system at the individual cell level, cellular distribution of BANG DNA was visualized using an anti-avidin antibody. Reporter gene expressions inside individual cells were also analyzed using anti-luciferase antibodies.

To investigate the in vivo behavior of the BANG system at the population cell level, transfection efficiency was. first examined using confocal microscopy and flow cytometery. The effect of the BANG system on the growth of the cells was also determined. Finally, transient and stable expressions of the BANG system were studied.

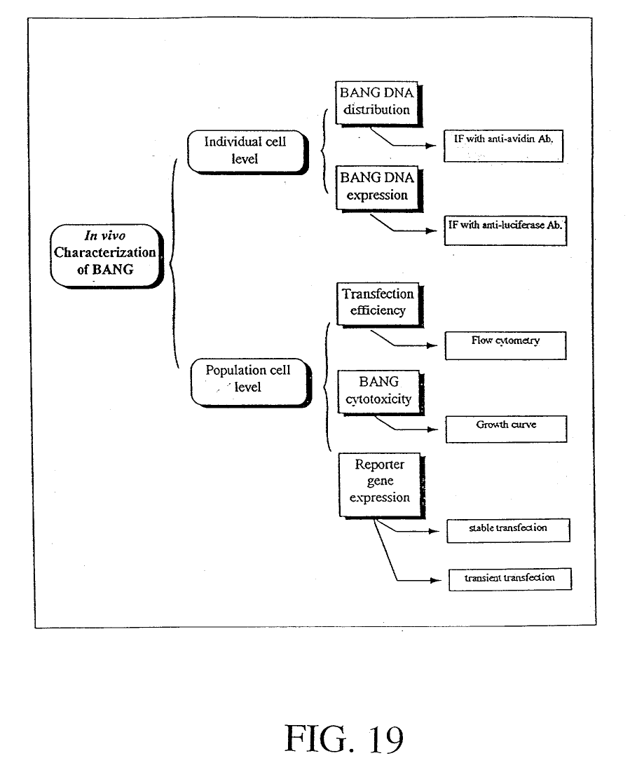

The strategies of in vivo characterization of the BANG system are illustrated in FIG. 19. Notice that mixtures of the monomer DNA and BANG D′TA were used in all the experiments (except where noted) in which the monomer DNA served as internal controls.

8.2 Materials and Methods

Plasmids and Primers

Green fluorescence protein (GFP) monomers were from PCR using a template of pGFP-C2 (Clonetech, Palo Alto, Calif. with two primers:

B-GFP-UP and B-GFP-DOWN, whose sequences were:

biotCTGATTCTGTGGATAACCGTATT, and

biotTGGAACAACACTCAACCCTATCT, respectively. Neomycine monomers were from PCR using the same template with two primers: B-GFP-UP and

B-GFP-DOWN, whose sequences were:

biotCTGATTCTGTGGATAACCGTATT, and

biotTGGAACAACACTCAACCCTATCT, respectively.

Immunostaining of BANG DNA distribution and luciferase expression in individual cells

Hela cells were transfected with mock and BANG DNA. Immunofluorescence microscopy was performed as described before (Example 2, Materials and Methods). A monoclonal anti-avidin antibody (Vector Laboratories, Inc. Burlingame, Calif.) was used as the primary antibody in the BANG DNA distribution study and a monoclonal anti-luciferase antibody (Promega, Madison, Wis.) was employed as the primary antibody in the luciferase expression study. A FITC-conjugated goat anti-mouse (GAM) secondary antibody was applied in both studies for viewing under an IF microscope.

Flow Cytometery Analysis

Flow analysis was performed on a Coulter EPICS Elite flow cytometer (Coulter Corporation, Miami, Fla.) equipped with a 488 nm 15 mW air-cooled Argon laser. Hela cells were first fixed in 20% cold ethanol (−20° C.) 48 hours after transfection. Anti-luciferase monoclonal antibody was then incubated with the fixed cells after washing in the PBS solution (without Ca++Mg++.). Cells were then labeled with FITC-conjugated GAM antibody-before flow analysis. Cells were gated from doublets, dead cells, and debris using linear forward light scatter versus 90° light scatter characteristics. A minimum of 5000 gated cells at a rate o f 500 events per second was collected. FITC fluorescence light emission was reflected through a 550 nm dichroic long pass filter and collected through a 525 nm band pass filter. The FITC signals were measure in logarithmic mode. The highest FITC intensity obtained from mock transfected cells was defined as the background threshold. Total cells were counted and their fluorescence was measured accordingly. The final results were analyzed using the Coulter Elite software.

Growth Curve of HL60

Suspension HL60 cells were cultured in RPMI medium supplemented with 10% fetal bovine serum (Hyclone Laboratories, Logan, Utah), and were transfected with mock, monomer DNA, or BANG DNA (the luciferase gene). Cell numbers were counted using a Hemocytometer at the specified times in triplicate. Trypan blue was used to stain and exclude dead cells.

Confocal Microscopy

A Maridian 750 ACAS confocal microscope was used to analyze Hela cells transfected with Green Fluorescence Protein BANG DNA. Cells were viewed directly without fixing and staining. Cells were scanned first by a laser (488 nm,) in single-scanning mode. The fluorescence intensities were then digitized and integrated for every single cell. The number of cells were plotted against the integrated fluorescence value (i.e., total fluorescence) in a histogram for each scanned field. w

Results

Cellular distribution of BANG DNA

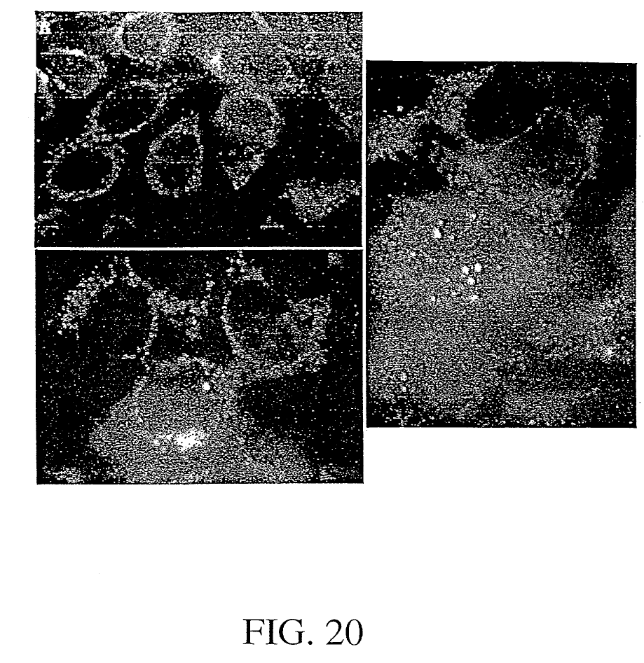

Large DNA networks such as kDNA had been successfully transfected into mammalian cells by lipofectamine in the previous experiments. BANG DNA were smaller in size than kDNA based on electrophoresis, and therefore it was expected that they would be able to be delivered into cells. To examine the cellular distribution of BANG DNA after transfection, a monoclonal anti-avidin antibody was applied to detect BANG DNA. The signal was visualized by a secondary antibody (goat anti-mouse) conjugated with FITC. FIG. 20 shows the cellular distribution of BANG DNA (panel B and C). Mock transfected cells were used as negative controls (panel A). The monomer control was not included since the anti-avidin antibodies did not recognize monomer DNA. Unlike kDNA whose distribution was mostly around the peripheral of the nucleus, the localization of BANG DNA was obviously concentrated inside the nucleus. Notice that the pictures presented here were selected for strong signals. About 100 cells were counted and the total stained cells were around 20%, indicating a reasonable transfection efficiency. This transfection efficiency was also seen in the subsequent experiments (see luciferase expression pictures and flow cytometry results, see below)

Luciferase Expression from BANG DNA in Individual Cells

To evaluate the in vivo expression potential of the BANG system, individual cells transfected with BANG DNA were examined by reporter-gene specific antibodies using IF procedures. Briefly, the luciferase gene under the control of the CMV promoter was amplified by PCR using biotinylated primers (see Chapter 6, Materials and Methods). BANG DNA was then formed by addition of avidin as described before (no seeding procedure). Fibroblast cells (NIH 3T3) were cultured in DMEM media supplement with 10% calf serum and were split onto glass coverslips in 60-mm dishes 24 hours before transfection. Lipofectamine was used according to the manufacturer's instructions for the transfection. Forty-eight hours after transfection, the coverslips were taken out for the IF procedure while the remaining cells were harvested for quantitative luciferase assays (next section). Cells were incubated with primary anti-luciferase antibody (monoclonal) after routine fixation and permeablization using 50% methanol and 50% acetone. Secondary antibodies (GAM) conjugated with FITC were then incubated with the cells before viewing under a Zeiss fluorescence microscope equipped with a CCD camera. IF pictures were taken along with phase contrast images.

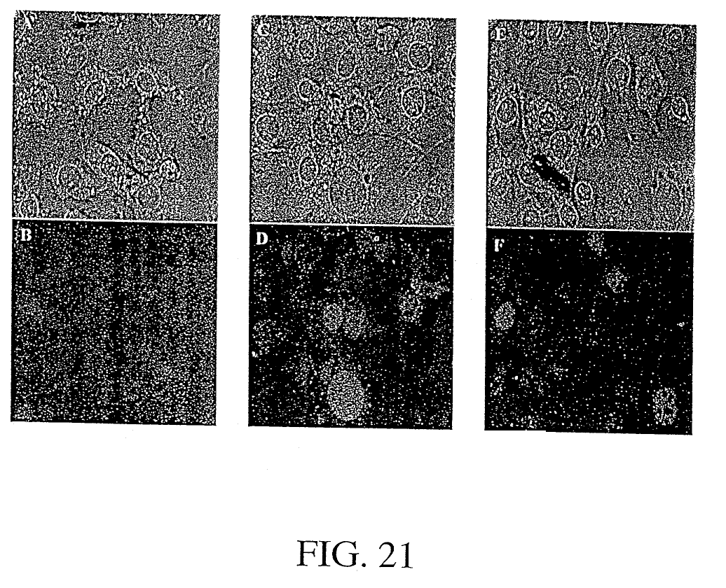

FIG. 21 contains the control IF pictures of luciferase expression in individual cells. Mock transfections gave only background signals (A and B). Two kinds of monomer DNA were used as controls: monomer DNA with no-biotinylation and no avidin incubations (C and D) shows a similar stain pattern as monomer DNA with biotinylation but without avidin incubations (E and F), indicating that neither biotinylation nor avidin incubations interfered with antibody specificity. Phase contrast images were used to monitor cell morphology and to identify fluorescence signals caused by debris.

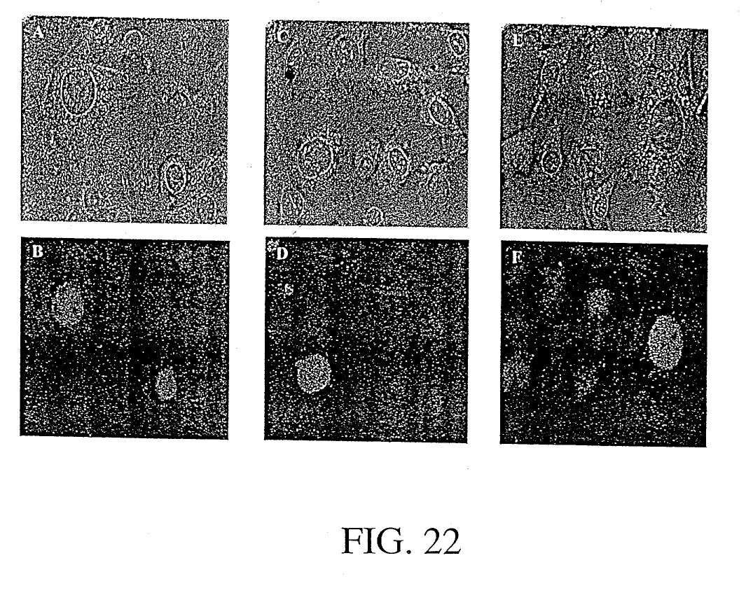

FIG. 22 shows the BANG DNA IF pictures of luciferase expression in individual cells. Because that BANG DNA used in this study was a mixture of monomer DNA and DNA-DNA crosslinked network, it was expected that many positive IF images should be similar to those obtained from pure monomer DNA. Comparing FIG. 22 with FIG. 21, it could be concluded that the expression patterns were comparable, at least to the naked eye.

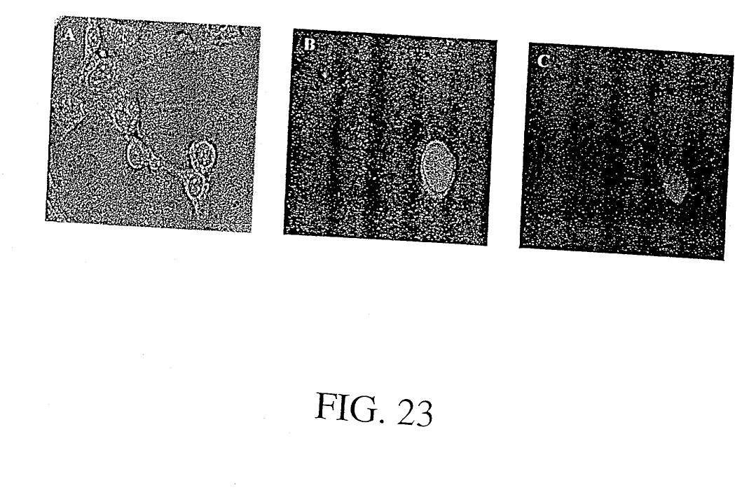

FIG. 23, on the other hand, reveals a striking over-expression cell achieved by BANG DNA transfection. Panels A, B, and C were pictures of the exact same field. An extremely bright signal was observed (panel B), and the phase contrast image (panel A) indicated that the signal was from a cell, not debris. The signal was so strong that it exceeded the linear range of exposure in that area. Using one tenth of the exposure time still saturated the exposure (data not shown). Using one hundredth of the exposure time (panel C) revealed a stained cell with a visible non-saturated signal. This suggested that the luciferase expression in that particular cell was at least 20-100 fold higher than most of the cells. Such intense signals were also observed in other fields of BANG DNA transfected cells (3 cells out of 150 total cells observed), but never in the fields of monomer transfected cells. In conclusion, the above results showed that over-expression of luciterase could be achieved in individual cells by BANG DNA transfection.

Transfection Efficiency

To evaluate transfection efficiency of both BANG DNA and monomer DNA, flow cytometry was applied. BANG DNA were generated through PCR method without the seeding procedure as described before. Lipofectamine was employed to deliver DNA. Transfected Hela cells were incubated for 48 hours before fixation and primary antibody (anti-luciferase) incubation. Secondary antibodies conjugated with FITC were then used for flowcytometry.

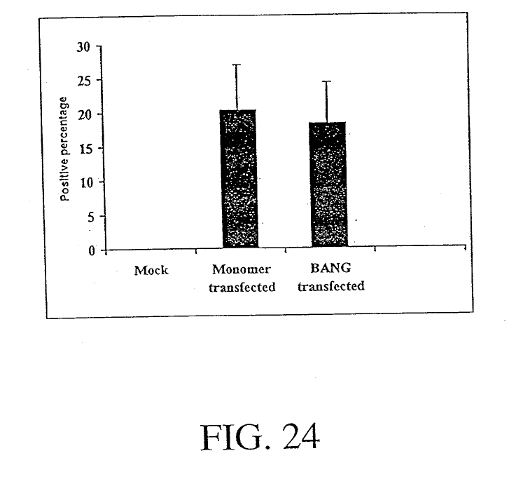

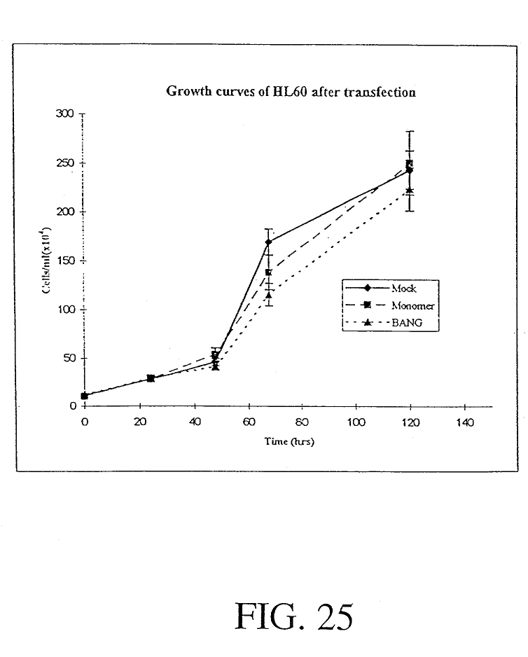

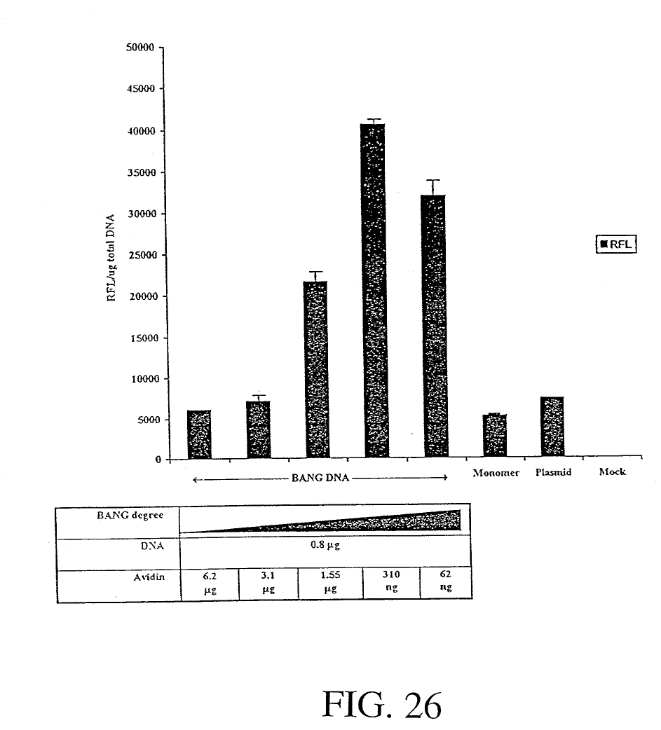

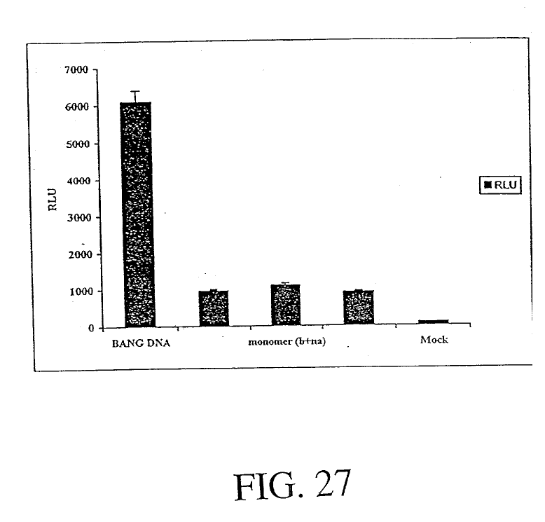

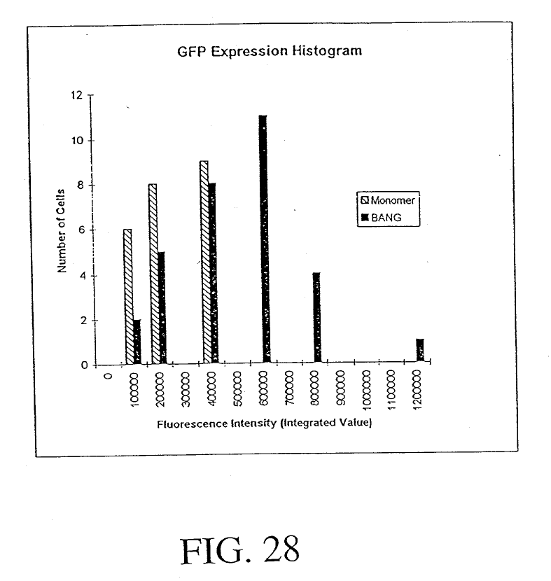

Flow analysis was performed on a Coulter EPICS Elite flow cytometer. Cells were gated from doublets, dead cells, and debris using linear forward light scatter versus 90° light scatter characteristics. A minimum of 5000 gated cells at a rate of 500 events per second were collected. The FITC signal was measured and analyzed using appropriate Coulter Elite software (Immuno-4 analysis). The, highest FITC intensity obtained from mock transfected samples was arbitrarily defined as a background (negative) threshold. Cells with FITC signals above that threshold were counted as positive cells. Total cells were then counted along with positive cells. The ratio of positive cell number over total cell number indicated transfection efficiency. From FIG. 24, it was obvious that the transfection efficiency was comparable between monomer DNA and BANG DNA.

A similar result was obtained by manually counting expressed cells using anti-luciferase antibodies (data not shown) after BANG DNA transfection. This was consistent with the notion that BANG DNA was able to be taken up by living cells using lipofectamine methods.

Cytotoxicity

To investigate the cytotoxicity of BANG DNA taken up by mammalian cells, growth curves of HL60 were constructed after transfection of BANG DNA A .(FIG. 25, triangles). Pure monomer DNA and mock transfections were used as controls (FIG. 25, squares and diamonds, respectively). The results (FIG. 25) clear indicated that the growth of HL60 cells did not seem to be inhibited by BANG DNA transfection over a period of 140 hours.

Stable Transfection of BANG DNA

In order to evaluate the long term expression ability of BANG DNA in mammalian cells, stable transfections were carried out. The Luciferase gene was used as a reporter gene, and the neomycine resistant gene was used as a drug selection marker. BANG DNA were formed by incubation of both biotinylated luciferase and neomycine resistant genes with avidin. NIH3T3 cells were about 20% confluent at the time of transfection. Monomer DNA were used as controls. Cells were incubated in a non-selective medium (DMEM with 10% calf bovine serum) for 48 hours before selective media (400 μg/ml G418) were applied. Colonies were formed after 36 days and cloned out for luciferase assays.

Eight colonies were formed in BANG DNA transfected cells, and 10 colonies were formed in monomer transfected cells. However, all of these colonies gave background level luciferase activities, indicating that the luciferase gene had not been integrated into their genome. There were several possible reasons for this negative result. It could be that the biotinylated DNA were difficult to integrate into the genome. It could also be that the DNA components in the BANG system were prone to nuclease digestion in vivo. At this point, it is difficult to explain the negative results. More experiments need to be done to further evaluate the ability of stable transfection by BANG DNA.

Transient Expression of Luciferase by BANG DNA in Hela Cells

FIG. 23 (above) reveals that over-expression of luciferase could be achieved in individual cells by BANG DNA transfection. To further evaluate such over-expression potentials at the population cell level, BANG DNA were transfected to Hela cells, along with control DNA (monomer DNA, supercoiled plasmid DNA). Luciferase activities were measured in terms of relative light unit (RLU), and were normalized to total cell numbers of each sample.

FIG. 26 shows the luciferase assay results 48 hours after transfection. Clearly, it shows that the luciferase activities correlate with the formation of BANG DNA. When Hela cells were transfected with little BANG DNA, the luciferase activities were very similar to those of cells transfected with monomer DNA or plasmid DNA. However, when there were more BANG DNA delivered to the Hela cells, over-expression of luciferase was observed: at least a 700% increase compared with those cells transfected with either monomer DNA or supercoiled plasmid DNA. Notice that BANG DNA used in this study were not purified, nor formed by the seeding procedure. In other words, the luciferase activities were contributed by both the monomer DNA and BANG DNA. Since the BANG DNA ratio was less than 50% of the total DNA (Example 2), the real increase of over-expression contributed by BANG DNA should be much higher. This was consistent with the previous results in which individual cells exhibited at least a 2000° increase in luciferase expression due to BANG DNA gene dosage effects.

Transient expression of luciferase by BANG DNA in NIH3T3 Cells

It could be argued that the increase of expression by BANG DNA might have been caused by increasing of transfection efficiency, not gene dosage effects of BANG DNA. It could also be argued that the increase of expression by BANG DNA might have been caused by avidin molecules in the BANG system, not BANG DNA per se. The following experiment was carried out to further confirm the transient expression results. It was designed to prove that, first, the over-expression of luciferase was indeed caused by BANG DNA, not because of the avidin molecules, nor due to the biotin molecules; and second, the results of higher transfection was cell-line independent, and not caused by higher transfection efficiency.

NIH3T3 cells were first seeded in a 35 mm culture dish containing one coverslip. Lipofectamine was used to transfect BANG DNA (DNA/avidin ratio: 0.8 μg/62 ng) along with various control DNA. Cells grown on the coverslip were then taken out for fixing and staining (with anti-luciferase antibodies). Transfection efficiency was obtained by counting the positively stained cells. The rest cells were harvested for luciferase assays. The RLU were normalized against the transfection efficiency.

FIG. 27 showed a consistent 600% increase in luciferase activities comparing BANG DNA transfected cells with monomer transfected cells. Notice that avidin molecules and DNA biotinylation did not seem to increase the. This again suggested that over-expression of BANG DNA was due to the gene dosage effect.

Transient expression of GFP in Hela cells using confocal microscopy

To further evaluate the over-expression characteristics of BANG DNA, a different reporter gene, Green Fluorescence Protein (GFP), was used. GFP is a non-catalytic, auto-fluorescent protein identified in the jelly fish Aquorea victoria. (Prasher, et al. 1992). It emits green light at a peak of 509 nm when absorbs blue light of 395 nm and 470 nm (Ward, et al., 1980). The advantage of using GFP was that the expression could be viewed live without fixing and staining cells. Confocal microscopy was applied in order to quantify expression of GFP in each individual cell.

FIG. 28 shows the composite of 6 different histograms. It revealed GFP expression at both population cell level and individual cell level. At the population cell level, GFP expression from the cells transfected with monomer DNA was clustered at average of about 200,000 units (hatched bars). No expression was higher than 400,000 units. However, GFP expression from the cells transfected with BANG DNA (mixture) showed a “bell” shape distribution with two populations: one similar to the monomer expression, and the other obviously over-expression (solid bars). At the individual cell level, half of the cells transfected with BANG DNA showed higher expression than those transfected with monomer DNA. Notice one cell exhibited at least 12 fold higher GFP expression than 6 cells received monomer DNA.

Discussion

Transfection Efficiency

All the evidence so far indicated that large DNA molecules, such as kDNA and BANG DNA, could be taken up by mammalian cells through lipofectamine transfection. In the case of kDNA, the distribution appeared to be around the peripheral of the nucleus. In the case of BANG DNA, on the other hand, the distribution seemed to be inside the nucleus.

The efficiency of transfection of BANG DNA and monomer DNA were quite similar. Even though the BANG DNA were not homogeneous (i.e., a mixture of BANG DNA and monomer), based on the results of this study, it was unlikely that the cells would selectively take up only monomer DNA (see DNA staining pattern in FIG. 20 and luciferase over expression in FIG. 23). However, exactly how cells took up BANG DNA via lipofectamine is still a mystery. It is also unknown whether or not there is an up-limit size of BANG DNA for cells to take up.

Over-expression

All the evidence so far revealed that BANG DNA could be over-expressed in mammalian cells due to the gene dosage effects. The over-expression seemed to be cell-line independent, and reporter-gene independent. Based on the in vitro and in vivo characterization of the BANG system, it is conceivable that BANG DNA could also be expressed in prokaryotic cells, such as E. Coli. Such investigations are underway.

The Upper Limit

This study clearly suggested that BANG DNA was able to over-express genes both at the individual cell level and at the population cell level, largely due to the gene dosage effect and reasonable transfection efficiencies. However, the up-limit level of over-expression still remains to be seen. It is a great challenge to define an up-limit level of over-expression due to the following reasons. First, the crosslinking patterns in BANG DNA are difficult to control. In other words, the networks are formed mainly by collision of biotinylated DNA and avidin molecules. The heterogeneous nature of BANG DNA also makes it difficult to be purified (even the same molecular weight does not guarantee the same crosslink pattern). Second, the biophysical behavior of BANG DNA is largely unknown. This gives rise to difficulties in identifying different BANG DNA. One may argue that the molecular weight could be used to distinguish one group of BANG DNA from another. The problem is that the same molecular weight (appears on the gel) does not necessarily mean the same BANG DNA, let alone the same expression level. Third, the pattern of expression is unclear. As can be seen in this study, while at the population cell level, BANG DNA exhibits at least a 7 fold increase in expression, at the individual cell level, the expression level could be much higher (at least 20 fold). Fourth, the upper limit is not only cell-line dependent, it is also reporter-gene dependent. The value is at least the combination of contributions of two factors: one, how much is expressed, and two, how much the cell can tolerate.

### Example 5

### Potential Applications of Bang and Conclusions Over Expression

The over-expression feature of the BANG system has been demonstrated throughout Example 4. This feature offers many advantages over conventional expression vectors, such as plasmids, thus leading to its great potentials.

In plasmids or other vectors, DNA sequences can only be linked by ligations (i.e. covalent linkages). However, in the BANG system, same or different DNA constellations could readily be brought together by biotin-avidin non-covalent interactions. This totally eliminates the reading-frame-shift and cloning problems. For example, a GFP gene and/or a neomycin resistant gene could easily be biotinylated and attached to any BANG system.

All plasmids (or other vectors) also need at least several kilobases of host sequences in order to maintain their propagation. For example, the amp gene usually is needed for maintaining strain purity, and the Ori sequence is needed for plasmid replications. The BANG system, however, is assembled totally in vitro, thus, effectively bypassing the bacterial/viral propagation cycles. This is a significant advantage, since only relevant DNA sequences are present in the over-expression system.

Manipulation of Bang DNA

The potentials of the BANG system also rely on the fact that the DNA-DNA network still possesses DNA characteristics. Combined with other DNA manipulation tools, such as ligases, endonucleases, restriction enzymes, DNA denaturelrenature etc., it should be very easy to modify the BANG system to researchers' own design.

Genegrid

In addition, by using the recycling GRASP procedure, a homogenous population of avidin saturated BANG DNA (GeneGrid) can readily be purified (Chapter 6). These cross-shaped DNA networks provide building blocks for the BANG system for further growth. The GRASP procedure is also adaptable to computer controlled automatic processes. Therefore, commercializing the BANG system along with the GeneGrid concept is in the foreseeable future.

It is anticipated that many important genes and sequences will be readily available in GeneGrid forms to researchers all over the world. For example, PromoterGrid,, which contains certain promoter sequences could be manufactured in advance. Ligases could then be used to attach the PromoterGrid to other BANG DNA for studying promoter functions and/or gene expression. GFPGrid, which contains green fluorescence proteins, could also be available for tracing other BANG system expressions without in-frame fusion protein cloning. Using such GeneGrid and the mix-match approaches the potential of the BANG system to bring different sequences together is beyond imagination.

DNA Vaccination

As revealed by the epidemic of the human immunodeficiency virus, there is no systematic method for producing a vaccine. Conventional procedures of generating vaccines usually either require live/attenuated pathogens or purification of the foreign proteins. DNA immunization was first proposed by Johnston (Tang, et al. 1992). Immune responses against a protein were elicited by introducing the gene coding for that protein into the skin of mice directly. Recently, it was also reported that injection of naked DNA into muscle cells (myocytes) could evoke long-lasting stable cellular and humoral immune responses (Huygen, et al. 1996; Tascon, et al. 1996; Waisman, et al., 1996).

The advantages of DNA vaccines are obvious: purity, ease of large scale production, stability, and most important of all, no risk of infection. The BANG system described in this dissertation enhances the potentials and scope of DNA vaccines. For example, the over-expression feature could increase the immune response. Multiple gene complexes could also be linked together via BANG to elicit broader immune reactions. Inducible gene expression assembled by BANG DNA might lead to inducible vaccines for target immunizations.

In collaboration with Dr. Glen Needham of the Ohio State University, the research of DNA vaccination using BANG DNA is in progress.

NON-covalent Cloning

Another exciting potential of the BANG system is the possibility of using the system as a cloning tool (i.e. non-covalent cloning). At present, genes have be to cloned first (used as templates in PCR) in order to utilize the BANG system. It should be possible, however, that the genes could be amplified randomly by PCR and assembled by the BANG system. Thus a “GeneGrid library” would be created.

Unlike the covalent cloning scheme in which the library is carried and maintained in phages or plasmids, the GeneGrid library does not need any host to maintain it. However, just as in the covalent cloning scheme, amplification and expression of such a library is essential in identifying a new gene. The exact ways of expressing and amplifying GeneGrid libraries are still under investigation. Nevertheless, the BANG system grants a possibility that not only a single gene, but also gene complexes can be cloned. This should greatly increase the understanding of the structure and function of different genes.

BANG Principle in other Macromolecules

Very recently, a paper published in Nature (Walker, et al. 1997) described the creation of vesosomes, a multicompartmental aggregate of tethered lipid vesicles encapsulated within a large lipid bilayer vesicle. The self-assembled vesosomes (liposomes within liposomes) were prepared using the molecular-recognition processes mediated by biotin-streptavidin complex. Combining the vesosome with BANG DNA may provide a new direction in drug delivery and gene therapy.

The BANG system is not limited to genes or DNA sequences. As a matter of fact, any macromolecule could be assembled into the BANG system as long as it could be biotinylated. For example, proteins could be easily biotinylated by N-hydroxysuccinimide (NHS)-biotin (Becker et al. 1971). Both carbohydrate and carboxyl groups of macromolecules could be targeted by biotin hydrazide (O'shannessy, et al. 1987. Wade et al. 1985). Furthermore, photoactivatabte biotin could be applied to label both DNA and RNA (Forster, et al., 1985). Biotin-labeled nucleic acids can also be prepared by the enzymatic incorporation of biotin-conjugated analogs of dUTP and UTP into DNA or RNA. Therefore, hybrid-macromolecules, such as protein-DNA or lipid-RNA may be produced by the BANG system.

Assembling New Organisms De Novo

Though it is conceivable that the BANG system may be used in the near future to create a huge network consisting of lipids, proteins, DNA and RNA. it is still an open question as to whether or not the BANG system can be used to assemble a totally new organism (de novo). In other words, bringing macromolecules together is one thing, making them work in concert and in an orderly way is a totally different matter. It is even more difficult to face the ethical, social and spiritual challenges ahead. As one of the inventors of the BANG technology, I would not want to see it used unethically and/or inhumanely.

Nevertheless, by opening a whole new field, the BANG system provides the first step of an exciting adventure. It has great potential in many scientific and clinical applications, such as over-expression, non-covalent cloning, gene-complex study, DNA vaccination, gene delivery, and gene therapy.

Conclusions

In this study, novel technologies were assessed to study protein-DNA crosslinking and DNA-DNA crosslinking.

A Single-pulsed high-power UV laser is capable of capturing the freeze frames of macromolecule interactions by instant covalent crosslinking of proteins and DNA. Therefore, this technology is well suited for the study of binding kinetics. The single-pulsed high-power UV laser was used to characterize the DNA binding kinetics of highly purified yeast topoisomerase II in the absence of ATP. It was also applied to identify the DNA-binding domain of topo II. At the mean time, a surface plasmon resonance (SPR) based biosensor technology, the BIAcore system, was also employed to determine the real time kinetics of DNA-binding properties of human topo II.

The yeast topo II DNA-binding affinity constant (K_{eq}) of 1.2±0.28×10⁸M⁻¹ was determined from laser crosslinking experiments. The human topo II DNA-binding affinity constant (K_{eq}) of 7.9±0.29×10⁷M⁻¹ was obtained from the BIAcore system. The effects of various clinically valuable topoisomerase drugs on the DNA binding constants of topo II were also investigated. Using limited digestion with V8 protease and peptide micro sequencing, the DNA-binding domain of yeast topo II was identified within a 29 kDa fragment with Leu-681 at its amino-terminal end.

Furthermore, a novel technology, the biotin-avidin networked gene (BANG) system, was devised to crosslink DNA-DNA molecules via non-covalent interactions. This system exploits the stability of biotin-avidin interactions, which is one of the strongest known non-covalent interactions between protein and ligand, to form networks between different segments of DNA. The formation of such branched and networked DNA molecules was confirmed by electrophoresis, transmission electron microscopy, and scanning electron microscopy. In vitro studies revealed that the BANG system was stable over protease treatment and a wide range of temperatures, and also accessible to proteins. In vivo characterization of the BANG system showed that the networks increased gene expression levels by at least 700% at the population cell level, and 2000% at the individual cell level.

The potential of the BANG system is enormous and exciting. It could be applied to much scientific and clinical research, such as over-expression, non-covalent cloning, gene-complex study, DNA vaccination, gene delivery, and gene therapy.

### BIBLIOGRAPHY

Adachi, Y. Käs, E. and Laemmli, U. K (1989) EMBO J. 8, 3997-4006

Ali, J. A. Jackson.A. P. HOWell, A. J. Maxwell, A- (1993a) Biochemistry 32, 2717-2724

Ali, J. A. Orphanides, G. Maxwell, A. (1993b) Biochemistry 34, 9801-9808

Amrein, M. Durr, R Stasiak, A- Gross, H. and Travaghni, G. (1989) Science 243, 1708-1711 Amrein, M. Durr. R. Stasiak, A. Gross, H. and Travaglini, G. (1989) Science 243,1708-1711 Amrein, M. Stasiak, A. Gross, H. Stoll, E. and Travaglini, G. (1988) Science 240, 514-516

Anderson, A. H. Christansen, K Zechiedrich, E. L. Jensen, P. S. Osheroff, N. and Westergaard, O. (1989) Biochemistry 28, 6237-6244

Anderson, H. J. and Roberge, M. (1992) Cell Biol. Int. Reports 16. 717-724

Angelov, D. Stefano vsky, V. Y. Dimintrov, S. I. Russanova, V. R. Keskinova, E. and Pashev, I. G. (1988) Nucleic Acids Res. 16, 4525-4538

Baldi, M. I., Benedetti, P., Mattoccia, E. and To cchini-Valentini, G. P. (1980) Cell, 20, 461-467

Bayer, E. A. and Wilchek, M. (1980) Meth. Biochem. anal, 26, 1-45

Becker, J. M. Wilchek, M. and Katchalski, E.(1971) Proc. Natl. Acad. Sci. USA 68, 2604-2607

Berger, J. M. Gamblin, S. Harrison, S. C. and Wang, J. C. (1996) Nature 379, 225-232

Berrios, M. Osheroff, N. and Fisher, P. A. (1985) Proc. Natl. Acad. Sci. USA 82, 4142-4146

Boas.M. A. (1927) Biochem. J. 21, 712-724

Bodley, A. L. and Liu, L. F. (1988) Biotechnology 6, 1315-1319

Bondesoa, K Frostell-Karlsson, Å. Fagerstam, L. and Magnusson, G. (1993) Anal. Chem. 214, 245-251

Buckle, M. Geiselmann, J. Kolb, A. and Buc, H. (1991) Nucleic Acids Res. 19, 833-840

Capramco, G. Kohn, K. W. and Pommier, Y. (1990a) Nucleic Acids Res. 18, 6611-6619

Capramco, G. Zunino, F. Kohn, K. W. and Pommier, Y. (1990) Biochemistry 29, 562-569

Careri, G. Fasella, P. and Gratton, E. (1975) CRC Crit. Rev. Biochem. 3, 141-164

Caron, P. R. and Wang, J. C. (1993) in: Molecular Biology of DNA Topoisomerases and Its Applications to Chemotherapy (Andoh, T Ikeda, H. and Oguro, M. eds) CRC Press, Boca Raton 1-18

Caron, P. R. and Wang, J. C. (1994) Adv. Pharmacol. 29B, 271-297

Chaiken, I. Rosé, S. and Karlsson, R. (1992)Anal. Chem. 201, 197-21.0

Chen, A. and Liu, L. F. (1994) Annu. Rev. Pharmacol.Toxicol. 34,191-218

Chen, G. L. Yang, L. Rowe, T. C. Halligan, B. D. Tewey, K M. and Liu, L. F. (1985) J. Biol. Chem., 259,13560-13566

Chen, J. Rauch, C. A. White, J. H. Englund, P. T. Cozzarelli, N. R. (1995) Cell 80, 61-69

C1eat, P. H. Hay, R. T. (1989) FEBS Lett. 258, 51-54

Cunningham, B. C. and Wells, J. A. (1993), J. Mol. Biol. 234, 554-563

DeLange, R. J. and Huang.T.-S. (1971) J. Biol. Chem. 246, 698-709

DiNardo, S. Voelkel, K and Sternglanz, R. (1984) Proc. Natl. Acad. Sci. USA 81, 2616-2620

Donovan, J. W. and Ross, K D. (1973) Biochemistry, 12(3), 512-517 du Vigneaud. V. Melville, D. B., György, P. and Rose, C. S. (1940) Science. 92, 62-63

Duhamel, R. and Whitehead, J. (1990) Methods in Enzymology, 184, 201-207

Earnshaw, W. C. Halligan, B. Cooke, C. A. Heck, M. S. and Liu, L. F. (1985). Cell Biol. 100, 1706-1716

Fagerstam, L. Frostell-Karisson, Å. Karlsson, R. Persson, B. and Rönnberg, I. (1992) J. Chromatogr. 597, 397-410.

Fisher, R. J. Fivash, M. Casa-Finet, J. Erickson, J. W. Kondoh, A. Bladen, S. V. fisher, C. Watson, D. K. and Paps, T. (1994), Protein Sci., 3, 257-266

Forster, A. C. McInnes, J. L. skingle, D. C. and Symons, R. H. (1985), Nucleic Acid Res. 13, 745-761

Glisson, B. S. and Ross, W. E. (1987) Pharmacol. Ther. 32, 89-106

Gope, M. L. Keinänen, R. A. Kristo, P. A. Conneely, O. M. Beattie, W. G. Zarucki-Schulz, T. O'Malley, B. W. and Kulomaa, M. S. (1987) Nucleic Acid Res. 15, 3595-360

Goto, T. and Wang, J. C. (1982) J. Biol. Chem., 257, 5866-5872

Goto, T. and Wang, J. C. (1984) Cell 36, 1073-1080

Green, N. M. (1966), Biochem. J. 101, 774-779

Green, N. M. (1975)Adv. Protein Chem. 29, 85-105

Green, N. M. Konieczny, L. Toms, E. J. and Valentine, R. C. (1971) Biochem. J. 125, 781-792

Green, N. M. and Melamed,M. D. (1966), Biochem. ,J. 100, 614-618

Green, N. M. and Toms, E. J. (1970) Biochem. J. 118, 67-79

György, P. Rose, C. S. Eakin, R. E. Snell, E. E. and Williams, R. J. (1941). Science 93. 477-478

György, P. Rose, C. S. Hofmann. K. Melville, D. B. and du Vigneaud,V. (1940) Science; 92, 609

Hiller, Y. Gershoni, J. M. Bayer, E. A. and Wilchek, M. (1987) Biochem. J. 248, 167-171

Ho, D. T. Sauvé, D. M. and Roberge, M. (1994) Anal. Biochem. 218,248-254

Hockensmith. J. W., Vorachek, W. R. Evertsz, E. M. and von Hippel, P. H. (1991) Methods Enzymol. 208, 211-236

Holm, C. Stearns, T. and Boststein, D. (1989) Mol. Cell. Biol. 9, 159-168

Howard, M. T. Griffith, J. D. (1991) J. Mol. Biol. 232, 1060-1068

Howard, M. T. Lee, M. P. Hsieh, T.-S. Griffith, J. D. (1991)J. Mol. Biol. 217, 53-62

Hsieh, T. and Brutlag, D. (1980) Cell, 21, 115-125

Hung, F. Luo, D. Sauvé, D. M. Muller, M. T. and Roberge, M. (1996) FEBS Lett. 380,127-132

Huygen, K Content, J. Denis, O. Montgomery, D. L. Yawman, A. M. Deck, R. R DeWitt, C. M. Orme, L M. Baldwin, S. D'Souza, C. Drowart, A. Lozes, E. Vandenbussche, P. Van Vooren, J. P. Liu, M. A. and Ulmer, J. B. (1996) Nature Medicine 2, 893-898

Johnsson, U. Fägerstam, L. Ivarsson, B. Johnsson, B. Karlsson, R Lundh, K. Lofas, S. Persson, B. Roos, H. Ronnberg, I. Sjolander, S. Sternberg, E. Stahlberg, R. Urbaniczcy, C. Ostlin, H. and Malmqvist, M. (1991), Biotechniques, 11, 620-627

Kretschmann, E. and Raether, H.(1968) Z Natuforschung, Teil. A, 230., 2135-2139

Kunkel, G. R. and Martinson, H. G. (1978) Nucleic Acids Res. 5, 4263-4272

Li, C. J. Dezube, B. J. Biswas, D. K Ahlers, C. M. and Pardee, A. B. (1994) Trends Microbiol. 2,164-169

Liu, L. F. (1989) Annu. Rev. Biochem. 58, 351-375

Lynen, F. Knappe, J. Lorch, E. Jutting, G. and Ringelmann, E. (1959), Angew. Chem. 71, 481-486

Lynn, R. Giaver, G. Swanberg, S. L. and Wang, J. C. (1986) Science, 233, 647-649

Marengere, L. E. M. Songyang, Z. Gish, G. P. Schaller, M. D. Parsons, J. T. Stern, M. J. Cantley, L. C. and Pawson, T. (1994), Nature 368, 502-505

Marini, J. C. Miller, K G. Englund, P. T. (1980) J. Biol. Chem. 255, 4976-4979

Markovitz, A. (1972) Biochem. Biophys. Res. Commun. 281, 522-534

Miller, K G. Liu, C. F. and Englund, P. T. (1981) J. Biol. Chem., 256, 9334-9339

Muller, M. T. Spitzner, J. R. DiDonato, J. A. Mehta, V. B. Tsutsui, K and Tsutsui, K (1988) Biochemistry 27, 8369-8379

Nilsson, P. Persson, B. Uhlën M. and Nygren. P.-A. (1995), Anal. Biochem. 224, 400-408

O'Shannessey, D. J. Brigham-Burke, M. and Peck, K. (1992) Anal. Biochem. 205,132-136

O'Shannessey, D. J. and Quarles, R. H. (1987) Anal. Biochem. 163, 204-209

Osheroff, N. (1986) J. Biol. Chem. 261, 9944-9950

Osheroff, N. (1987) Biochemistry 26, 6402-6406

Osheroff, N. (1989) Pharmacol. ther. 41, 223-241

Osheroff, N. Shelton E. R. and Brutlag, D. L. (1983) J. Biol. Chem. 258, 9536-9543

Osheroff, N. Zechiedrich, E. L. and Gale, K. C. (1991) BioEssays 13, 269-275

Otto, A. (1968) Pkys. Stat. Solidi. 26, 199-203

Park, C. S. Hillel, Z. and Wu, C. W. (1980) NucleicAcids Res. 8, 5895-5912

Pashev, I. G., Dimitrov, S. I. and Angelov, D. (1991) Trends Biol. Sci. 16, 323-326.

Pommier, Y. Kerrigan D. and Kohn K. (1989) Biochemistty 28, 995-1002

Prasher, D. C. Eckenrode, V. K. Ward, W. W. Prendergast, F. G. and Cormier, M. J. (1992) Gene 111, 229-233

Rauch, C. A. Pérez-Morga, D. Cozzarelli, N. R. Englund, P. T. (1993) EMBO J. 12,403-411

Robinson, M. J. and Osheroff, N. (1990b) Biochemistry 30, 1807-1813

Robison, M. J. and Osheroff N. (1990a) Biochemistry 29, 2511-2515

Roca, J. and Wang, J. C. (1992) Cell 71, 833-840

Roca, J. Berger, J. M. Wang, J. C. (1993) J. Biol. Chem. 268,14250-14255

Rose, D. Thomas, W. and Holm C. (1990) Cell 60, 1009-1017

Rowe, T. C. Chen, G. L. Hsiang, Y.-H. and Liu, L. F. (1986) Cancer Research 46, 2021-2026

Sambrook, J. Fritsch E. F. and Maniatis, T. (1989) Molecular Cloning: a Laboratory Manual, 2nd edn., Cold Spring Harbor University Press., Cold Spring Harbor, N.Y.

Sambrook, J. Fritsch, E. F. and Maniatis, T. (1989) Molecular Cloning: a Laboratory Manual, 2nd edn., Cold Spning Harbor University Press, Cold Spring Harbor, N.Y.

Sander, M. and Hsieh, T.-S. (1983) J. biol. Chem. 258, 8421-8428

Sander, M. and Hsieh, T.-S. (1985) Nucl. Acids Res. 13, 1057-1072

Sander, M. Hsieh., T. Udvardy, A. and Schedl.P (1987) J. Mol. Biol. 194 (2), 219-229

Shiozaki, K and Yanagida, M. (1991) Mol. Cell. Biol. 11, 6093-6102

Spitzner, J. R and Muller, M. T. (1988) Nucleic Acids Res. 16, 5533-5556

Spitzner, J. R. Chung, I. K. and Muller, M. T. (1990) Nucleic Acids Res. 18, 1-11

Spitzner, J. R., and Muller, M. T. (1989) J. Mol. Recognit. 2, 63-74.

Sterniste, G. F. and Smith, D. A. (1974) Biochemistry 13, 485-492

Tang, D. C. De Vit, M. and Johnston, S. A. (1992) Nature, 356,152-154

Tascon, R. E. Colston, M. J. Ragno, S. Stavropoulos, E. Gregory, D. and Lowrie, D. B. (1996), Nature Medicine. 2, 888-892

Turbadar, T. (1959) Proc. Phys. Soc. (London) 73, 40-43

Uemura, T. and Yanagida, M. (1984) EMBO. J. 3, 1737-1744

Uemura, T. Ohkura, H. Adachi, Y. Morino, K Shiozaki, K and Yanagida, M. (1987); Cell 50, 917-925

Wade, D. P. Knight, B. L. and Soutar, A. K. (1985) Biochem. J. 229, 785-790

Waisman, A. Ruiz, P. J. Hirschberg, D. L. Gelman, A. Oksenberg, J. R. Brocke, S. Mor, F. Cohen, I. R. and Steinman, L. (1996) Nature Medicine 2, 899-905

Wakil, S. J. Titchener, E. B. and Gibson, D. M. (1958), Biochim. Biophy. Acta 29, 225-226

Walker, S. A. Kennedy, M. T. and Zasadzinski, J. A. (1997) Nature, 387, 61-64

Wang, J. C. (1985) Ann. Rev. Biochem. 54, 665-697

Wang, J. C. (1996) Ann. Rev. Biochem. 65, 635-692

Ward, W. W. Cody, C. W. Hart, R. C. and Cormier, M. J. (1980) Photochem. Photobiol. 31, 611-615

Wilchek, M. and Bayer, E. A. (1990), Methods in Enzymology 184,14-45

Zechidrich, E. L., Christiansen, K., Andersen, A. H., Westgaard, O. and Osheroff. N. (1988) Biochemistry 28, 6229-6236

Zechiedrich, E. L. Osheroff, N. (1990) EMBO J. 9, 4555-4562

All books, articles and patents cited in this specification are incorporated by reference in their entirety.

While the present invention has been described in connection with what is presently considered to be practical and preferred embodiments, it is understood that the present invention is not to be limited or restricted to the disclosed embodiments but, on the contrary, is intended to cover various modifications and equivalent arrangements included within the spirit and scope of the appended claims.

Thus, it is to be understood that variations in the described invention will be obvious to those skilled in the art without departing from the novel aspects of the present invention and such variations are intended to come within the scope of the claims below.

1. A composition comprising complexes which comprise polynucleotide molecules covalently coupled to ligand moieties, said ligand moieties being specifically bound to ligand-binding sites of ligand-binding molecules in said complexes, wherein:
each of said polynucleotide molecules is covalently coupled to at least one of said ligand moieties;
each of said ligand-binding molecules comprises more than one of said ligand-binding sites; and
the number of said polynucleotide molecules in said complexes is
equal to at least about 50% of the total number of said ligand-binding sites of all of said ligand-binding molecules in said complexes and
greater than about 50% of all polynucleotide molecules in said composition.

2. A composition of claim 1 wherein further the number of said polynucleotide molecules specifically bound to said ligand-binding sites in said complexes is greater than about 80% of all polynucleotide molecules in said composition.

3. A composition of claim 1 wherein further said polynucleotide molecules in said complexes comprise linear polynucleotide molecules and a 5′ end of each of said linear polynucleotide molecules is covalently coupled to one of said ligand moieties.

4. A composition of claim 1 wherein further said polynucleotide molecules in said complexes comprise single-stranded polynucleotide molecules.

5. A composition of claim 1 wherein further said polynucleotide molecules in said complexes comprise polynucleotide molecules which are at least partially double-stranded.

6. A composition of claim 1 wherein further said polynucleotide molecules in said complexes comprise nucleotides selected from the group consisting of deoxyribonucleotides, ribonucleotides, analogs of deoxyribonucleotides, and analogs of ribonucleotides.

7. A composition of claim 1 wherein further said ligand moieties and said ligand-binding molecules are selected from the group consisting of the following pairs of ligand moieties and ligand-binding molecules:
an antigen moiety and an antibody or fragment thereof which specifically binds to said antigen moiety;
an oligosaccharide moiety and a lectin-binding protein or fragment thereof which specifically binds to said oligosaccharide moiety;
an enzyme inhibitor moiety and an enzyme or fragment thereof which specifically binds to said enzyme inhibitor moiety; and
a biotin moiety and a biotin-binding protein or fragment thereof which specifically binds to said biotin moiety.

8. A composition of claim 1 wherein further said ligand moieties are covalently coupled to said polynucleotide molecules by a linker moiety.

9. A composition of claim 1 wherein further:
said polynucleotide molecules comprise linear single-stranded DNA molecules,
each of said DNA molecules is covalently coupled to one of said ligand-moieties which is covalently coupled to the 5′ end of each of said DNA molecules; and
each of said ligand-binding molecules comprises four of said ligand-binding sites.

10. A composition of claim 9 wherein further:
said ligand moieties comprise biotin moieties and said ligand-binding sites comprise biotin-binding sites.

11. A composition of claim 1 wherein further:
said polynucleotide molecules comprise linear DNA molecules which are at least partially double-stranded, and
each of said DNA molecules is covalently coupled to one of said ligand moieties which is covalently coupled to the 5′ end of one strand of said DNA molecules.

12. A composition of claim 11 wherein further the number of said polynucleotide molecules specifically bound to said ligand-binding sites in said complexes is greater than about 80% of all polynucleotide molecules in said composition.

13. A composition of claim 11 wherein further:
each of said ligand-binding molecules comprises four of said ligand-binding sites.

14. A composition of claim 13 wherein further:
said ligand moieties comprise biotin moieties and said ligand-binding sites comprise biotin-binding sites.

15. A composition of claim 1 wherein further said polynucleotide molecules encode a polypeptide.

16. A composition of claim 15 wherein further said polynucleotide molecules encode a transcriptional unit comprising a sequence encoding said polypeptide.

17. A composition of claim 1 wherein further said polynucleotide molecules encode a sequence of at least ten nucleotides, said sequence being complementary to at least ten nucleotides of a nucleotide sequence encoding a transcriptional unit.

18. A method of making a composition of claim 1, said method comprising:
contacting said ligand-binding molecules with a sample of said polynucleotide molecules under conditions such that said ligand-binding sites on said ligand binding molecules bind specifically to said ligand moieties which are covalently coupled to said polynucleotide molecules,
wherein the total number of said ligand-binding sites of all of said ligand-binding molecules contacted with said sample is less than the number of said ligand moieties coupled to said polynucleotide molecules in said sample.

19. A method according to claim 18, wherein said total number of said ligand-binding sites contacted with said sample is at least about ten times less than the number of said ligand moieties coupled to said polynucleotide molecules in said sample.

20. A method according to claim 18, said method further comprising:
after contacting said ligand-binding molecules with said sample, removing from said sample some of said polynucleotide molecules covalently coupled to ligand moieties that are not bound to said ligand-binding sites of said ligand-binding molecules in said sample.

21. A method according to claim 20 wherein said polynucleotide molecules covalently coupled to said ligand moieties that are not bound to said ligand-binding sites in said complexes are removed from said sample by
contacting said sample with a solid support, said solid support being coated with ligand-binding molecules having ligand-binding sites specific for said ligand moiety, under conditions such that said ligand moieties that are not bound to said ligand-binding sites in said complexes specifically bind to ligand-binding molecules on said solid support, and
separating said complexes remaining in said sample from said solid support.

22. A method of making a composition of claim 1 wherein further:
said polynucleotide molecules comprise linear DNA molecules which are at least partially double-stranded, and
each of said DNA molecules is covalently coupled to one of said ligand moieties which is covalently coupled to the 5′ end of one strand of said DNA molecules, said method said method comprising:
successively contacting small amounts of said ligand-binding molecules with a sample of said DNA molecules under conditions such that said ligand-binding sites on said ligand binding molecules bind specifically to said ligand moieties which are covalently coupled to said polynucleotide molecules,
wherein the total number of said ligand-binding sites of all of said ligand-binding molecules in each of said small amounts contacted with said sample is less than the number of said ligand moieties coupled to said DNA molecules in said sample.

23. A method according to claim 22, wherein said total number of said ligand-binding sites in each of said small amounts contacted with said sample is at least about 100 times less than the number of said ligand moieties coupled to said polynucleotide molecules in said sample.

24. A method of delivering polynucleotide molecules to a viable cell comprising contacting a composition of claim 1 with said viable cell.

25. A method according to claim 24, wherein said complexes in said composition which is contacted with said viable cell are contained in liposomes.

26. A method according to claim 24 wherein said complexes further comprise a component which enhances uptake of said polynucleotides in said complexes.

27. A method according to claim 26 where said component which enhances uptake of said polynucleotides is selected from the group consisting of a cation, a ligand moiety which specifically binds to a receptor that undergoes endocytosis, a peptide comprising a nuclear localization sequence, a peptide comprising a cellular membrane fusion sequence, and an endosome-disruptive peptide.
